(19) United States
(12) Patent Application Publication
(10) Pub. No.: US 2014/0037615 A1
(43) Pub. Date: Feb. 6, 2014

(54) MATERIALS AND METHODS FOR DESIGNING AUTOLOGOUS IDIOTYPE VACCINES AND TREATMENT OF B-CELL MALIGNANCIES

(71) Applicants: Carlos F. Santos, TAMPA, FL (US); Mihaela Popa-Mckiver, Valrico, FL (US); Amy M. McCord, New Port Richey, FL (US); Mark Hirschel, Blaine, MN (US)

(72) Inventors: Carlos F. Santos, TAMPA, FL (US); Mihaela Popa-Mckiver, Valrico, FL (US); Amy M. McCord, New Port Richey, FL (US); Mark Hirschel, Blaine, MN (US)

(73) Assignee: BIOVEST INTERNATIONAL, INC., TAMPA, FL (US)

(21) Appl. No.: 13/816,918

(22) PCT Filed: Aug. 12, 2011
(86) PCT No.: PCT/US2011/047645

§ 371 Date: Sep. 23, 2013

Related U.S. Application Data
(60) Provisional application No. 61/373,735, filed on Aug. 13, 2010.
(60) Provisional application No. 61/411,453, filed on Nov. 8, 2010.
(60) Provisional application No. 61/420,243, filed on Dec. 6, 2010.

(51) Int. Cl. A61K 39/395

(52) U.S. Cl. CPC A61K 39/39566
USPC 424/131.1; 435/7.92

(57) ABSTRACT

The present invention relates to compositions, kits, and methods for preparing an autologous idiotype vaccine, or autologous anti-idiotype vaccine, for treatment of a B-cell malignancy based on the isotype(s) (class(es)) of immunoglobulins expressed by the malignancy; methods for treating B-cell malignancies; and methods for selecting a treatment for a subject having a B-cell malignancy.

### CROSS-REFERENCE TO RELATED APPLICATIONS

This application claims the benefit of U.S. Provisional Application Ser. No. 61/373,735, filed Aug. 13, 2010, U.S. Provisional Application Ser. No. 61/411,453, filed Nov. 8, 2010, and U.S. Provisional Application Ser. No. 61/420,243, filed Dec. 6, 2010, the disclosure of each of which is incorporated herein by reference in its entirety, including all figures, tables and drawings.

### BACKGROUND OF THE INVENTION

Surgery, chemotherapy and radiation therapy are the mainstay of cancer treatment and management. Surgery and radiation therapy are typically used to achieve results locally, whereas chemotherapy exerts a more systemic effect. However, usually remaining cancer cells are able to divide, thereby leading to a relapse of the cancer. Accordingly, despite the use of combination chemotherapy to treat various types of cancers, a significant number of cancers remain incurable.

More recently, immunotherapeutic strategies have been developed for the treatment of various cancers. The central premise underlying immunotherapy for cancer is the presence of antigens that are selectively or abundantly expressed or mutated in cancer cells. Tumor-specific immunotherapies can be classified into passive immunotherapy with antibodies targeted directly to tumor cells or active immunotherapy via vaccination with tumor cells, tumor cell lysates, peptides, carbohydrates, genetic constructs encoding proteins, or anti-idiotypic antibodies that mimic tumor-associated antigens (TAA). The ideal cancer vaccine both targets an antigen that is uniquely expressed on the cancer cells and overcomes the immune system's existing tolerance to that antigen.

The collection of unique antigenic epitopes formed by the variable regions of the heavy and light chains of immunoglobulins is called the idiotype (Id). Vaccination with anti-Id antibodies relies on the observation that the variable antigen-binding regions of antibodies contain Id determinants that are immunogenic and induce the formation of anti-Id immunoglobulins. A subset of these antibodies is able to functionally mimic the three-dimensional structure of the original antigen. Consequently, selective immunization with idiotype antibodies can induce a specific immune response against the original antigen (Jefferis, R., “What is an idiotype?” Immunol. Today, 1993, 14:119-121; Chatterjee, M. et al., “Idiotypic antibody immunotherapy of cancer” Cancer Immunol. Immunother., 1994, 38:75-82). B-cells express clonal immunoglobulin with unique idiotypes, and T cell receptors possess unique idiotypes. When T-cells and B-cells undergo malignant transformation, these idiotypes can serve as unique tumor targets for immune therapy.

### BRIEF SUMMARY OF THE INVENTION

Tumor-derived idiotype (Id) protein conjugated to keyhole limpet hemocyanin (KLH) administered with granulocyte-monocyte colony-stimulating factor (GM-CSF) can induce follicular lymphoma (FL)-specific immune responses that target tumor-specific antigenic deteiminants within the tumor cell's unique immunoglobulin (Ig) variable region (Fv). While Fv idiotypic determinants serve as specific tumor antigens, preclinical evidence suggests that the isotype of the Ig constant region (Fc) may independently influence the immunogenicity of hybridoma-derived immunoglobulins. Whereas Ids that have switched to IgG were tolerogenic, Ids of their IgM progenitors were highly immunogenic (Reitan et al. Proc Natl Acad Sci USA, 2002). Thus, the present inventors examined the clinical impact of tumor Ig isotype on disease-free survival (DFS) within a prospective randomized double-blind placebo-controlled multicenter phase III study of patient-specific tumor heterohybridoma-derived Id vaccine in advanced stage previously untreated FL patients with a lymph node adequate for vaccine production.

Among 76 patients receiving Id vaccine, 36 received IgM-Id vaccines and 40 IgG-Id vaccines corresponding to their tumor Ig isotype. Of 41 patients receiving control, 25 had tumors with IgM isotype and 15 had tumors with IgG isotype; 1 patient had a tumor with mixed IgM/IgG isotypes. Among 36 patients with IgM tumor isotype receiving an IgM-Id vaccine, median time to relapse after randomization was 50.6 months, versus 27.1 months in the IgM tumor isotype control-treated patients (log-rank p=0.002; HR=0.36 (p=0.003); [95% CI: 0.19-0.71]) (shown in FIG. 2). Among 40 patients with IgG tumor isotype receiving an IgG-Id vaccine, median time to relapse after randomization was 35.1 months, versus 32.4 months in control-treated patients with IgG tumor isotype (log-rank p=0.807; HR=1.1 (p=0.807); [95% CI: 0.50-2.44]) (shown in FIG. 3). It must be noted that although this trial was not powered to address such subsetting, the dramatically different results suggest that the treatment effect is different in the two groups, with a surprisingly small p-value of 0.085.

These results suggest that the isotype of an Id vaccine may influence DFS in FL patients vaccinated in first complete remission. Unexpectedly, it was observed that the IgM-Id vaccine significantly prolonged remission duration in comparison to IgG-Id vaccine. Compared to other phase III Id vaccine trials that used recombinant Id vaccines with IgG constant regions for all patients, the positive outcome of our study may reflect the use of hybridomas to produce Id protein with variable and constant regions identical to patient tumor Ig. Additional studies are expected to further evaluate the effect of Id vaccine isotype on clinical outcome in FL and other B-cell malignancies. These findings should have profound implications on Id vaccine production strategies and clinical development.

One aspect of the invention concerns a method for preparing an autologous idiotype vaccine for treatment of a B-cell malignancy in a subject in which the immunoglobulin isotype or isotypes exhibited by the malignancy has been predetermined, wherein the method comprises preparing an autologous idiotype vaccine for the subject, and wherein the vaccine comprises an idiotype immunoglobulin comprising at least an IgM constant region.

Another aspect of the invention concerns a method for treating a B-cell malignancy in a subject in which the immunoglobulin isotype or isotypes exhibited by the malignancy have been predetermined, comprising administering an autologous idiotypic vaccine to the subject, wherein the vaccine comprises an autologous idiotype immunoglobulin comprising at least an IgM constant region.

Another aspect of the invention concerns a method for selecting a treatment for a subject having a B-cell malignancy, comprising screening the subject for a heavy-chain isotype, wherein if the isotype has detectable M isotype (for example, IgM, IgM+IgG, or IgM+Ig* (wherein * is any isotype) production of an autologous idiotype IgM vaccine is authorized and treatment of the subject with the autologous idiotype IgM vaccine can proceed; and wherein if the subject has only a non-IgM B-cell malignancy, (a) production of a recombinant idiotype vaccine for the subject (idiotype+IgM) is authorized and treatment of the subject with the recombinant vaccine can proceed; or optionally (b) the subject is excluded from treatment with an idiotype vaccine and an alternative treatment with an alternative (non-idiotype vaccine) therapy is authorized (for example, rituximab+chemotherapy: R-CHOP, R-CVP or PACE, or a chlorambucil-containing chemotherapy, or autologous stem cell transplant) and may proceed.

In some embodiments of the methods of the invention, one or more booster doses of the autologous idiotype vaccine are administered to the subject, e.g., about 24 months to about 30 months after completion of the initial treatment with the vaccine. In some embodiments, the booster doses of the autologous idiotype vaccine are administered to the subject about 24 months to about 30 months after completion of the initial treatment and administered again in about 12 to about 18 months thereafter. In some embodiments, the booster doses of the autologous idiotype vaccine are administered to the subject about 24 months to about 30 months after completion of the initial treatment and administered again in about 12 to about 18 months thereafter, and periodically at about every 12 to 18 months thereafter.

In some embodiments of the methods of the invention, the initial treatment is for a B-cell malignancy (also referred to herein as a B-cell derived malignancy).

Examples of B-cell malignancies include non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, Burkitt lymphoma/leukemia.

In some embodiments, the B-cell malignancy is a mature B-cell lymphoma. Examples of mature B-cell lymphomas include B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, Burkitt lymphoma/Burkitt cell leukemia.

The mature B-cell lymphoma may be a variant malignancy, for example, B-cell chronic lymphocytic leukemia/small lymphocytic lymphoma with monoclonal gammopathy/plasmacytoid differentiation, hairy cell leukemia variant, cutaneous follicle center lymphoma, diffuse follicle center lymphoma, blastoid mantle-cell lymphoma, morphologic variant of diffuse large B-cell lymphoma (for example, centroblastic, immunoblastic, T-cell/histiocyte-rich, lymphomatoid granulomatosis type, anaplastic large B-cell, plasmablastic) or subtype of diffuse large B-cell lymphoma (for example, mediastinal (thymic) large B-cell lymphoma, primary effusion lymphoma, intravascular large B-cell lymphoma), morphologic variant of Burkitt lymphoma or Burkitt cell leukemia (for example, Burkitt-like lymphoma/leukemia, Burkitt lymphoma/Burkitt cell leukemia with plasmacytoid differentiation (AIDS-associated), or clinical or genetic subtype of Burkitt lymphoma/Burkitt cell leukemia (for example, endemic, sporadic, immunodeficiency-associated).

In some embodiments of the methods of the invention, the autologous idiotype vaccine comprises an antigen associated with a B-cell derived malignancy in the subject, and wherein the antigen is produced by a hybridoma (for example, by hybridoma rescue fusion hybridization, see, e.g., Lee, G. and B. Ge, “Inhibition of in vitro tumor cell growth by RP215 monoclonal antibody and antibodies raised against its anti-idiotype antibodies,” Cancer Immunol Immunother, 59(9): p. 1347-1356; Thanavala, Y. M., et al., “Monoclonal ‘internal image’ anti-idiotypic antibodies of hepatitis B surface antigen Immunology,” 55(2): 197-204 (1985)). In some embodiments, the hybridoma is produced by fusion of a cancerous B-cell obtained from the subject and a murine/human heterohybridoma myeloma cell (for example, the K6H6/B5 cell line). In some embodiments, the antigen-producing hybridoma is grown in a hollow-fiber bioreactor. The immunoglobulin can then be collected from the hollow-fiber bioreactor and purified (for example, by affinity chromatography) prior to administration to the subject.

Preferably, in both the initial treatment and the one or more booster doses (if booster doses are given), the purified antigen is conjugated to a carrier molecule, such as an immunogenic carrier protein (for example, keyhole limpet hemocyanin (KLH)) or other immunogenic carrier molecule, prior to administration to the subject.

Preferably, in the initial treatment, the autologous idiotype vaccine is administered in conjunction with an effective amount of an adjuvant, such as granulocyte monocyte-colony stimulating factor (GM-CSF). In some embodiments, the one or more booster doses of the autologous idiotype vaccine are administered without an adjuvant.

The initial treatment with the autologous idiotype vaccine can comprise one or more administrations. Preferably, the initial treatment is a regimen comprising a plurality of administrations of the autologous idiotype vaccine. In some embodiments, the initial treatment comprises five administrations of the autologous idiotype vaccine over a period of about 6 months. In some embodiments, the autologous idiotype vaccine comprises an antigen associated with a B-cell derived malignancy in the subject, and a carrier molecule linked to the antigen, and the initial treatment comprises administration (e.g., subcutaneous) of 0.01 mg to about 100 mg of the autologous idiotype vaccine (day 1) and about 50 μg/m²/day to about 200 μg/m²/day granulocyte monocyte-colony stimulating factor (days 1-4) at about 1, 2, 3, 4, and 6 months. In some embodiments, the autologous idiotype vaccine comprises an antigen associated with a B-cell derived malignancy in the subject, and keyhole limpet hemocyanin linked to the antigen, and the initial treatment comprises administration (e.g., subcutaneous) of 0.5 mg of the autologous idiotype vaccine (day 1) and 100 μg/m²/day granulocyte monocyte-colony stimulating factor (days 1-4) at about 1, 2, 3, 4, and 6 months.

In some embodiments, the booster dose comprises about 0.01 mg to about 100 mg autologous idiotype vaccine per administration (e.g., subcutaneous). In some embodiments, the booster dose comprises about 0.5 mg autologous idiotype vaccine per administration (e.g., subcutaneous).

In some embodiments, the subject has undergone a different therapy (i.e., other than the autologous idiotype vaccine therapy) prior to the initial treatment with the vaccine, such as chemotherapy and/or immunotherapy. In some embodiments, the different therapy comprises therapy with a monoclonal antibody, such as rituximab, tositumomab, ibritumomab tiuxetan, or epratuzumab (see, for example, Cheson B. D. and J. P. Leonard, N, Engl. J. Med., 359(6):613-626 (2008)). In some embodiments, the different therapy comprises a radioimmunotherapy, such as ibritumomab tiuxetan. In some embodiments, the different therapy comprises a regimen of PACE (prednisone, doxorubicin, cyclophosphamide, and etoposide) or CHOP-R (cyclophosphamide, hydroxydaunrubicin, oncovin, prednisone/prednisolone, and rituximab). Preferably, the different therapy induces complete remission in the subject prior to the initial treatment with the vaccine. Preferably, the subject is in complete remission at the time of the initial treatment with the vaccine. Preferably, the subject is in complete remission at the time that each of the one or more booster doses of the vaccine is administered.

Another aspect of the invention provides a method for maintaining a sustained immune response against a B-cell idiotype in a subject, the method comprising: (a) administering an effective amount of an autologous idiotype vaccine to the subject; and (b) administering at least one booster dose of the autologous idiotype vaccine to the subject. Preferably, the administering of (a) induces an immune response against a B-cell idiotype in the subject. Preferably, the immune response comprises both a cellular and humoral immune response. In some embodiments, the administering of at least one booster dose of (b) is conducted at least about 20 months after the administering of (a). In some embodiments, the at least one booster dose of (b) is administered to the subject about 24 months to about 30 months after the administering of (a). In some embodiments, the at least one booster dose of (b) is administered to the subject about 24 months to about 30 months after the administering of (a), and administered again in about 12 to about 18 months thereafter. In some embodiments, the at least one booster dose of (b) is administered to the subject about 24 months to about 30 months after the administering of (a), and administered again in about 12 to about 18 months thereafter, and periodically at about every 12 to 18 months thereafter.

In some embodiments, the administering step of (a) is for treatment of a B-cell derived malignancy, such as non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, Ill, or IV), diffuse large B-cell lymphoma, mediastinal (thymic) large B-cell lymphoma, intravascular large B-cell lymphoma, primary effusion lymphoma, and Burkitt lymphoma/leukemia.

In some embodiments, the autologous idiotype vaccine administered in (a) and (b) comprises an antigen associated with a B-cell derived malignancy in the subject, and the antigen is produced by a hybridoma. In some embodiments, the hybridoma is produced by fusion of a cancerous B-cell obtained from the subject and a murine/human heterohybridoma myeloma cell, such as the K6H6/B5 cell line, or a cell line such as 1D12. In some embodiments, the antigen-producing hybridoma is grown in a hollow-fiber bioreactor. The antigen can then be collected from the hollow-fiber bioreactor and purified (e.g., by affinitiy chromatography) prior to administration to the subject. Preferably, in the administering steps of (a) and (b), the purified antigen is linked to a carrier molecule such as an immunogenic carrier protein (e.g., KLH) prior to administration to the subject.

Preferably, in the administering step of (a), the autologous idiotype vaccine is administered to the subject in conjunction with an effective amount of an adjuvant, such as GM-CSF. In some embodiments, the one or more booster doses of (b) are administered without an adjuvant.

The administering step of (a) can comprise one or more administrations of the autologous idiotype vaccine. Preferably, the administering step of (a) is a regimen comprising a plurality of administrations of the autologous idiotype vaccine. In some embodiments, the administering step of (a) comprises five administrations of the autologous idiotype vaccine over a period of about 6 months. In some embodiments, the autologous idiotype vaccine comprises an antigen associated with a B-cell derived malignancy in the subject, and a carrier molecule linked to the antigen, and the initial treatment comprises administration (e.g., subcutaneous) of 0.01 mg to about 100 mg of the autologous idiotype vaccine (day 1) and 50 μg/m²/day to about 200 μg/m²/day granulocyte monocyte-colony stimulating factor (days 1-4) at about 1, 2, 3, 4, and 6 months. In some embodiments, the autologous idiotype vaccine comprises an antigen associated with a B-cell derived cancer in the subject, and keyhole limpet hemocyanin linked to the antigen, and wherein said administering of (a) comprises administration (e.g., subcutaneous) of 0.5 mg of the autologous idiotype vaccine (day 1) and 100 μg/m²/day granulocyte monocyte-colony stimulating factor (days 1-4) at about 1, 2, 3, 4, and 6 months. In some embodiments, the booster dose(s) of step (b) comprises 0.01 mg to about 100 mg autologous idiotype vaccine per administration (e.g., subcutaneous). In some embodiments, the booster dose(s) of (b) comprises about 0.5 mg autologous idiotype vaccine per administration (e.g., subcutaneous).

In some embodiments, the subject has undergone a different therapy (i.e., other than the autologous idiotype vaccine therapy) prior to the administering of step (a), such as chemotherapy and/or immunotherapy. In some embodiments, the different therapy comprises therapy with a monoclonal antibody, such as rituximab, tositumomab, ibritumomab tiuxetan, or epratuzumab. In some embodiments, the different therapy comprises a radioimmunotherapy, such as ibritumomab tiuxetan. In some embodiments, the different therapy comprises a regimen of PACE (prednisone, doxorubicin, cyclophosphamide, and etoposide) or CHOP-R (cyclophosphamide, hydroxydaunrubicin, oncovin, prednisone/prednisolone, and rituximab). Preferably, the different therapy induces complete remission in the subject prior to the administering step of (a). Preferably, the subject is in complete remission at the time of the administering of (a). Preferably, the subject is in complete remission at the time that each of the one or more booster doses is administered in (b).

Another aspect of the invention provides a method for maintaining an immune response against a B-cell idiotype in a subject, the method comprising: (a) administering an effective amount of an autologous idiotype vaccine to the subject such that an immune response against the B-cell idiotype is induced; (b) assessing an immune response to the autologous idiotype vaccine in the subject and determining whether the immune response against the vaccine has diminished; and (c) administering at least one booster dose of the autologous idiotype vaccine to the subject if the immune response against the vaccine is determined to have diminished. In some embodiments, assessing of the immune response to the autologous idiotype vaccine of (b) comprises assessing the immune response against the B-cell idiotype. In some embodiments, the autologous idiotype vaccine comprises an antigen associated with a B-cell derived cancer in the subject, wherein the antigen is linked to a carrier molecule, and wherein assessing of the immune response to the autologous idiotype vaccine of (b) comprises assessing the immune response to the carrier molecule. In some embodiments, assessing of the immune response to the autologous idiotype vaccine of (b) comprises both assessing the immune response against the B-cell idiotype and assessing the immune response against the carrier molecule. In some embodiments, the determining of (b) comprises comparing the immune response as assessed after the administering of (a) to a prior or subsequent assessment of the immune response in the subject. In some embodiments, assessing of the immune response to the autologous idiotype vaccine of (b) is carried out multiple times at uniform or non-uniform time intervals after the administering of (a), and wherein the determining of (b) comprises comparing two or more of the multiple assessments to determine whether the immune response to the autologous idiotype vaccine has diminished. In some embodiments, the at least one booster dose of (c) is administered to the subject, and wherein the method further comprises administering at least one additional booster dose of the autologous idiotype vaccine to the subject if the immune response to the autologous idiotype vaccine is determined to have diminished since the at least one booster dose of (c).

The present invention provides methods of treating various B-cell derived malignancies and, in particular, B-cell derived cancers, such as, for example, non-Hodgkin's lymphoma, Hodgkin's lymphoma, chronic lymphocytic leukemia, mantle cell lymphoma or multiple myeloma, using an autologous idiotype vaccine.

In one aspect of the present invention, a method of eliminating or substantially reducing non-Hodgkin's lymphoma in a subject is provided. The method includes administering an effective amount of an autologous idiotype tumor vaccine, thereby to eliminate or substantially reduce non-Hodgkin's lymphoma in the subject and re-administering an effective amount of the autologous idiotype tumor vaccine (as a booster dose), thereby to maintain the elimination or substantial reduction of non-Hodgkin's lymphoma (e.g., to achieve and maintain complete clinical remission (no clinically detectable signs of disease)). In some embodiments, the booster dose(s) of the autologous idiotype vaccine is administered at least about 20 months after the initial administration. In some embodiments, the booster dose(s) of the autologous idiotype vaccine is administered to the subject about 24 months to about 30 months after completion of the first administration. In some embodiments, the booster doses of the autologous idiotype vaccine are administered to the subject about 24 months to about 30 months after completion of the first administration and administered again in about 12 to about 18 months thereafter. In some embodiments, the booster doses of the autologous idiotype vaccine are administered to the subject about 24 months to about 30 months after completion of the first administration and administered again in about 12 to about 18 months thereafter, and periodically at about every 12 to 18 months thereafter.

In another aspect of the present invention, a method of eliminating or substantially reducing Hodgkin's lymphoma in a subject is provided. The method includes administering an effective amount of an autologous idiotype tumor vaccine, thereby to eliminate or substantially reduce Hodgkin's lymphoma in the subject, and re-administering an effective amount of the autologous idiotype tumor vaccine, thereby to maintain the elimination or substantial reduction of Hodgkin's lymphoma (e.g., to achieve and maintain complete clinical remission (no clinically detectable signs of disease)). In some embodiments, the booster dose(s) of the autologous idiotype vaccine is administered at least about 20 months after the initial administration. In some embodiments, the booster dose(s) of the autologous idiotype vaccine is administered to the subject about 24 months to about 30 months after completion of the first administration. In some embodiments, the booster doses of the autologous idiotype vaccine are administered to the subject about 24 months to about 30 months after completion of the first administration and administered again in about 12 to about 18 months thereafter. In some embodiments, the booster doses of the autologous idiotype vaccine are administered to the subject about 24 months to about 30 months after completion of the first administration and administered again in about 12 to about 18 months thereafter, and periodically at about every 12 to 18 months thereafter.

In yet another aspect of the present invention, a method of eliminating or substantially reducing chronic lymphocytic leukemia (CLL) in a subject is provided. The method includes administering an effective amount of an autologous idiotype tumor vaccine, thereby to eliminate or substantially reduce chronic lymphocytic leukemia in the subject, and re-administering an effective amount of the autologous idiotype tumor vaccine, thereby to maintain the elimination or substantial reduction of CLL (e.g., to achieve and maintain complete clinical remission (no clinically detectable signs of disease)). In some embodiments, the booster dose(s) of the autologous idiotype vaccine is administered at least about 20 months after the initial administration. In some embodiments, the booster dose(s) of the autologous idiotype vaccine is administered to the subject about 24 months to about 30 months after completion of the first administration. In some embodiments, the booster doses of the autologous idiotype vaccine are administered to the subject about 24 months to about 30 months after completion of the first administration and administered again in about 12 to about 18 months thereafter. In some embodiments, the booster doses of the autologous idiotype vaccine are administered to the subject about 24 months to about 30 months after completion of the first administration and administered again in about 12 to about 18 months thereafter, and periodically at about every 12 to 18 months thereafter.

In a further aspect of the present invention, a method of eliminating or substantially reducing mantle cell lymphoma in a subject is provided. The method includes administering an effective amount of an autologous idiotype tumor vaccine, thereby to eliminate or substantially reduce mantle cell lymphoma in the subject, and re-administering an effective amount of the autologous idiotype tumor vaccine, thereby to maintain the elimination or substantial reduction of mantle cell lymphoma (e.g., to achieve and maintain complete clinical remission (no clinically detectable signs of disease)). In some embodiments, the booster dose(s) of the autologous idiotype vaccine is administered at least about 20 months after the initial administration. In some embodiments, the booster dose(s) of the autologous idiotype vaccine is administered to the subject about 24 months to about 30 months after completion of the first administration. In some embodiments, the booster doses of the autologous idiotype vaccine are administered to the subject about 24 months to about 30 months after completion of the first administration and administered again in about 12 to about 18 months thereafter. In some embodiments, the booster doses of the autologous idiotype vaccine are administered to the subject about 24 months to about 30 months after completion of the first administration and administered again in about 12 to about 18 months thereafter, and periodically at about every 12 to 18 months thereafter.

In yet another aspect of the present invention, a method of eliminating or substantially reducing multiple myeloma in a subject is provided. The method includes administering an effective amount of an autologous idiotype tumor vaccine, thereby to eliminate or substantially reduce multiple myeloma in the subject, and re-administering an effective amount of the autologous idiotype tumor vaccine, thereby to maintain the elimination or substantial reduction of multiple myeloma (e.g., to achieve and maintain complete clinical remission (no clinically detectable signs of disease)). In some embodiments, the booster dose(s) of the autologous idiotype vaccine is administered at least about 20 months after the initial administration. In some embodiments, the booster dose(s) of the autologous idiotype vaccine is administered to the subject about 24 months to about 30 months after completion of the first administration. In some embodiments, the booster doses of the autologous idiotype vaccine are administered to the subject about 24 months to about 30 months after completion of the first administration and administered again in about 12 to about 18 months thereafter. In some embodiments, the booster doses of the autologous idiotype vaccine are administered to the subject about 24 months to about 30 months after completion of the first administration and administered again in about 12 to about 18 months thereafter, and periodically at about every 12 to 18 months thereafter.

In one or more aspects of the present invention, a method for eliminating or substantially reducing non-Hodgkin's lymphoma or Hodgkin's lymphoma or chronic lymphocytic leukemia, mantle cell lymphoma or multiple myeloma further includes administration of an effective amount of granulocyte-monocyte colony stimulating factor (GM-CSF). In some embodiments, GM-CSF is administered in conjunction with an autologous idiotype vaccine.

In another aspect of the present invention, a method for eliminating or substantially reducing a B-cell derived cancer selected from the group consisting of non-Hodgkin's lymphoma, Hodgkin's lymphoma, chronic lymphocytic leukemia, mantle cell lymphoma and multiple myeloma is provided. The method includes administering an effective amount of an autologous anti-idiotype anti-tumor vaccine in conjunction with granulocyte-monocyte colony stimulating factor to the subject, thereby to eliminate or substantially reduce the B-cell derived cancer, and re-administering an effective amount of the autologous anti-idiotype anti-tumor vaccine. In one embodiment, the autologous anti-idiotype anti-tumor vaccine is administered without granulocyte-monocyte colony stimulating factor.

In the methods and compositions of the invention, the IgM constant region may be that of human or humanized immunoglobulins, and may be recombinant or non-recombinant (recombinantly produced or non-recombinantly produced). Preferably, in cases in which the subject is human, the IgM constant region utilized is human or humanized.

In some embodiments, the methods of the invention comprise administering a T-regulatory cell inhibitor to the subject, and subsequently administering an idiotype vaccine to the subject.

Another aspect of the invention features a method for selecting a treatment for a B-cell malignancy, comprising determining the T-regulatory (T-reg) cell level (T-reg cell number and/or T-reg activity) in the subject; wherein if the T-reg cell level is consistent with a normal T-reg cell level, an effective amount of a T-reg cell inhibitor is administered to the subject prior to administration of a vaccine of any preceding claim. The T-reg cell level can be determined by obtaining one or more biological samples from the subject (for example, blood, peripheral blood, synovial fluid, or other biological tissue or fluid where T-reg cells are found) and determining the T-reg cell level in the sample(s) prior to administration of a vaccine of the invention. Preferably, the T-reg cell inhibitor is administered to the subject until the T-reg cell level in the subject is below that of a threshold, immunosuppressive T-reg cell level. In some embodiments, the T-reg cell level is determined two or more times and the T-reg cell inhibitor is administered to the subject until the T-reg cell level in the subject is below that of a threshold, immunosuppressive T-reg cell level, prior to administration of the vaccine. T-reg cell level can be determined by methods known in the art. For example, T-reg cells in a sample can be quantitated by flow cytometry. Sub-populations of T-reg cells can be targeted for level determination, such as CD4+ CD25HIFoxp3+ cells.

Another aspect of the invention relates to autologous idiotype vaccines comprising at least an IgM constant region and compositions containing the vaccines. In some embodiments, the composition further comprises one or more anti-cancer compounds.

Another aspect of the invention features a kit for treatment of a B-cell malignancy, comprising at least one autologous idiotype vaccine and printed instructions for using the vaccine for treatment of the B-cell malignancy. In some embodiments, the kit further comprises an immune adjuvant and/or one or more reagents for assessing immune response in a subject. In some embodiments, the idiotype vaccine comprises an autologous idiotype immunoglobulin linked to a carrier molecule.

Another aspect of the invention features a kit for assessing a humoral response to a vaccine of the invention, comprising an assay for detection of anti-idiotype immunoglobulins in a sample through their capacity to bind to the vaccine idiotype; and printed instructions for using the assay to detect the humoral response. The assay may be an enzyme-linked immunsorbent assay (ELISA), for example. The assay can be a colorimetric, chemiluminescent, fluorescent, or radioactive assay, for example.

Another aspect of the invention features a kit for assessing a cellular response to a vaccine of the invention, comprising an assay for detection of one or more activation markers, cytokines, growth factors, or cell subsets indicative of a cellular response, or a combination of two or more of the foregoing. The assay may be an enzyme-linked immunsorbent assay (ELISA), for example. The assay can be a colorimetric, chemiluminescent, fluorescent, or radioactive assay, for example.

Another aspect of the invention features a kit for detecting the T-regulatory (T-reg) cell response before, during, and after administration of a T-reg cell inhibitor prior to administration of a vaccine of the invention, wherein the kit comprises one or more reagents for assessing T-reg cell response in a subject; and printed instructions for making the assessment. In some embodiments, the kit further comprises a T-reg cell inhibitor.

### BRIEF DESCRIPTION OF THE FIGURES

FIGS. 1A and 1B are, respectively, a clinical trial schema and flow chart of enrollment, randomization, and treatment. As shown in FIG. 1A, advanced stage, previously untreated, follicular lymphoma patients underwent a lymph node biopsy (LN Bx) after enrollment and were treated with prednisone (60 mg/m2 orally daily on days 1 to 14), doxorubicin (25 mg/m2 IV on days 1 and 8), cyclophosphamide (650 mg/m2 IV on days 1 and 8), and etoposide (120 mg/m2 IV on days 1 and 8) (PACE) chemotherapy every 28 days. Patients achieving a complete response (CR)/complete response unconfirmed (CRu) were stratified according to International Prognostic Index (IPI) and number of chemotherapy cycles and randomized 2:1 to receive five injections of the Id-vaccine (Id-KLH+GM-CSF) or control vaccine (KLH+GM-CSF), respectively. As shown in FIG. 1B, two hundred thirty-four patients were enrolled and 117 patients were randomized to receive at least one dose of the blinded vaccine; 76 received Id-vaccine and 41 received control vaccine. Patients receiving fewer than 5 immunizations either withdrew from the study; or relapsedt before completion.

FIGS. 2A and 2B are graphs showing disease-free survival (DFS) and overall survival (OS) according to treatment group for the randomized patients that received blinded vaccinations (N=117). Kaplan-Meier survival curves for DFS (FIG. 2A) and OS (FIG. 2B) for randomized patients who received at least one dose of Id-vaccine (N=76; red) or control vaccine (N=41; blue) are shown. The number of events, median, and 95% confidence intervals for each group are also presented.

FIGS. 3A and 3B are graphs showing DFS according to tumor immunoglobulin (Ig) heavy chain isotype for the randomized patients that received blinded vaccination. Randomized patients who received at least one dose of the Id-vaccine or control vaccine were grouped according to the isotype of their tumor Ig heavy chain. Kaplan-Meier survival curves for DFS for Id-vaccine (red) and control vaccine (blue) groups according to IgM (FIG. 3A) and IgG (FIG. 3B) isotype are shown. The number of events, median DFS, and 95% confidence intervals for each group are also presented.

FIG. 4 is a graph showing DFS according to treatment group for all randomized patients (N=177). Kaplan-Meier survival curves for DFS for all randomized patients are shown according to treatment group: Id-vaccine (N=118; red); control (N=59; blue). The number of events, median DFS, and 95% confidence intervals for each group are also presented.

FIG. 5 is a graph showing DFS according to treatment group for randomized patients who did not receive vaccination (N=60). Kaplan-Meier survival curves for DFS for randomized patients who did not receive vaccination are shown according to treatment group: Id-vaccine (N=42; red); control (N=18; blue). The number of events, median DFS, and 95% confidence intervals for each group are also presented.

FIG. 6 is a graph showing DFS for the randomized patients that received IgM-Id vaccine versis all controls. Kaplan-Meier survival curves for DFS for the IgM-Id vaccinated patients (N=35; red) and all patients in the control arm (N=41; blue) are shown. The number of events, median DFS, and 95% confidence intervals for each group are also presented.

### DETAILED DESCRIPTION OF THE INVENTION

### Definitions

In order that the present disclosure may be more readily understood, certain terms are first defined. Additional definitions are set forth throughout the detailed description.

The terms “eliminating,” “substantially reducing,” “treating,” and “treatment,” as used herein, refer to therapeutic or preventative measures described herein. The methods of “eliminating or substantially reducing” employ administration to a subject having a B-cell malignancy. In some embodiments, the term “eliminating” refers to a complete remission of a B-cell malignancy in a subject treated using the methods described herein.

The terms “B lymphocyte” and “B cell,” as used interchangeably herein, are intended to refer to any cell within the B cell lineage as early as B cell precursors, such as pre-B cells B220′ cells which have begun to rearrange Ig VH genes and up to mature B cells and even plasma cells such as, for example, plasma cells which are associated with multiple myeloma. The term “B-cell,” also includes a B-cell derived cancer stem cell, i.e., a stem cell which is capable of giving rise to non-Hodgkin's lymphoma, Hodgkin's lymphoma, chronic lymphocytic leukemia, mantle cell lymphoma or multiple myeloma. Such cells can be readily identified by one of ordinary skill in the art using standard techniques known in the art and those described herein.

The terms “B-cell malignancy” and “B-cell derived malignancy” refer to a malignancy arising from aberrant replication of B cells. B-cell malignancies include, for example, non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, Burkitt lymphoma/leukemia. The B-cell malignancy may be a mature B-cell lymphoma. Examples of mature B-cell lymphomas include B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, Burkitt lymphoma/Burkitt cell leukemia.

The mature B-cell lymphoma may be a variant malignancy, for example, B-cell chronic lymphocytic leukemia/small lymphocytic lymphoma with monoclonal gammopathy/plasmacytoid differentiation, hairy cell leukemia variant, cutaneous follicle center lymphoma, diffuse follicle center lymphoma, blastoid mantle-cell lymphoma, morphologic variant of diffuse large B-cell lymphoma (for example, centroblastic, immunoblastic, T-cell/histiocyte-rich, lymphomatoid granulomatosis type, anaplastic large B-cell, plasmablastic) or subtype of diffuse large B-cell lymphoma (for example, mediastinal (thymic) large B-cell lymphoma, primary effusion lymphoma, intravascular large B-cell lymphoma), morphologic variant of Burkitt lymphoma or Burkitt cell leukemia (for example, Burkitt-like lymphoma/leukemia, Burkitt lymphoma/Burkitt cell leukemia with plasmacytoid differentiation (A/DS-associated), or clinical or genetic subtype of Burkitt lymphoma/Burkitt cell leukemia (for example, endemic, sporadic, immunodeficiency-associated).

The terms “immunoglobulin” and “antibody” (used interchangeably herein) include a protein having a basic four-polypeptide chain structure consisting of two heavy and two light chains, said chains being stabilized, for example, by interchain disulfide bonds, which has the ability to specifically bind an antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind an antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable,” based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions.” The “constant” domains of an antibody light chain are referred to interchangeably as “light chain constant regions,” “light chain constant domains,” “CL” regions or “CL” domains. The “constant” domains of an antibody heavy chain are referred to interchangeably as “heavy chain constant regions,” “heavy chain constant domains,” “CH” regions or “CH” domains). The “variable” domains of an antibody light chain are referred to interchangeably as “light chain variable regions,” “light chain variable domains,” “VL” regions or “VL” domains). The “variable” domains of an antibody heavy chain are referred to interchangeably as “heavy chain constant regions,” “heavy chain constant domains,” “VH” regions or “VH” domains).

Immunoglobulins or antibodies can exist in monomeric or polymeric form, for example, IgM antibodies which exist in pentameric form and/or IgA antibodies which exist in monomeric, dimeric or multimeric form. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

The term “antigen-binding portion” of an antibody (or “antibody portion”) includes fragments of an antibody that retain the ability to specifically bind to an antigen (e.g., a B-cell specific antigen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P. et al., (1993) Proc. Natl. Acad. Sci. USA 90:6444-6448; Poljak, R. J. et al., (1994) Structure 2:1121-1123). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M. et al., (1995) Human Antibodies and Hybridomas 6:93-101) and use of a cysteine residue, a marker peptide and a C-terminal polyhistidine tag to make bivalent and biotinylated scFv molecules (Kipriyanov, S. M. et al., (1994) Mol. Immunol., 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

“Specific binding,” “specifically binds,” “specific for”, “selective binding,” and “selectively binds,” as used herein, mean that the compound, e.g., antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). For example, in one embodiment, an antibody or antigen-binding portion thereof, that specifically binds to a B-cell specific antigen, such as, for example, CD-20 or CD-22, will appreciably bind CD-20 or CD-22, but will not significantly react with other non-CD-20 or non-CD-22 proteins or peptides. Specific or selective binding can be determined according to any art-recognized means for determining such binding, including, for example, according to Scatchard analysis and/or competitive binding assays.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The term “human antibody” includes antibodies having variable and constant regions corresponding to human germline immunoglobulin sequences as described by Kabat et al. (See Kabat, et al., (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In a preferred embodiment, these replacements are within the CDR regions as described in detail below.

The term “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D. et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat E. A., et al., (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody” includes an antibody that is substantially free of other antibodies having different antigenic specificities (e.g., an isolated antibody that specifically binds a B-cell specific antigen and is substantially free of antibodies or antigen-binding portions thereof that specifically bind other antigens, including other B-cell antigens). An isolated antibody that specifically binds a B-cell specific antigen may bind the same antigen and/or antigen-like molecules from other species. Moreover, an isolated antibody may be substantially free of other cellular material and/or chemicals.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The terms “idiotype,” “Id,” and “idiotypic determinant,” as used herein, refer to an epitope in the hypervariable region of an immunoglobulin. Typically, an idiotype or an epitope thereof is formed by the association of the hypervariable or complementarity determining regions (CDRs) of VH and VL domains.

The terms “anti-idiotype” and “anti-Id,” refer to an antibody, or antigen-binding portion thereof, that binds one or more idiotypes present on an antibody.

The term “autologous idiotype vaccine” refers to a composition, the active ingredient of which is an immunogenic molecule that is preferably capable of inducing an immune response against a B-cell idiotype derived from the same subject to which it is administered. In some embodiments, the immunogenic molecule in a vaccine used in the methods of the present invention is a normal product of a subject's B cells that happens to be expressed clonally on the cancer cells (e.g., cells derived from a Hodgkin's lymphoma or non-Hodgkin's lymphoma or chronic lymphocytic leukemia, mantle cell lymphoma or multiple myeloma) and serves as a unique a target for immune attack. In some embodiments, the vaccine comprises an IgM anti-Id immunoglobulin. In some embodiments, an “autologous idiotype vaccine,” is capable of eliciting an immune response against a B-cell idiotype derived from a subject having non-Hodgkin's lymphoma. In another embodiment, an “autologous idiotype vaccine,” is capable of eliciting an immune response against a B-cell idiotype derived from a subject having Hodgkin's lymphoma. In yet another embodiment, an “autologous idiotype vaccine,” is capable of eliciting an immune response against a B-cell idiotype derived from a subject having chronic lymphocytic leukemia. In a further embodiment, an “autologous idiotype vaccine,” is capable of eliciting an immune response against a B-cell idiotype derived from a subject having multiple myeloma. In a yet further embodiment, an “autologous idiotype vaccine,” is capable of eliciting an immune response against a B-cell idiotype derived from a subject having mantle cell lymphoma. In some embodiments of the present invention, an “autologous idiotype vaccine,” is used for the treatment of a B-cell derived cancer in combination with other immune therapeutics such as, for example, monoclonal antibodies that selectively bind B-cell specific antigens. In some embodiments, an “autologous idiotype vaccine” includes an antigen associated with a B-cell derived cancer in a subject (e.g., non-Hodgkin's lymphoma, Hodgkin's lymphoma, chronic lymphocytic leukemia, mantle cell lymphoma or multiple myeloma) linked to KLH (keyhole limpet hemocyanin, a carrier protein). In some embodiments of the present invention, an autologous idiotype vaccine is administered in conjunction with GM-CSF, and subsequently re-administered, as a booster, one or times with or without GM-CSF.

The term “granulocyte monocyte colony stimulating factor” or “GM-CSF” refers to a hematopoeitic growth factor that stimulates the development of committed progenitor cells to neutrophils and enhances the functional activities of neutrophils. It is produced in response to specific stimulation by a variety of cells including macrophages, fibroblasts, endothelial cells and bone marrow stroma. Either purified GM-CSF or recombinant GM-CSF, for example, recombinant human GM-CSF (R & D SYSTEMS, INC, Minneapolis, Minn.) or sargramostim (LEUKINE, BAYER HEALTHCARE Pharmaceuticals, Wayne, N.J.) can be used in the methods described herein.

The phrase “an effective amount of granulocyte monocyte colony stimulating factor” refers to an amount of granulocyte monocyte colony stimulating factor, which upon a single or multiple dose administration to a subject, induces or enhances an immune response in the subject (e.g., as an adjuvant). In some embodiments, 50 μg/m²/day to about 200 μg/m²/day (e.g., 100 μg/m²/day) granulocyte monocyte colony stimulating factor is administered to the subject. In some embodiments, “an effective amount of granulocyte monocyte colony stimulating factor” refers to a daily administration of 5 μg/kg of the granulocyte colony stimulating factor.

As used herein, the term “antigen” refers to a molecule (for example, a polypeptide, nucleic acid molecule, carbohydrate, glycoprotein, lipid, lipoprotein, glycolipid, or small molecule) that is capable of eliciting an immune response and contains an epitope or antigenic determinant to which an immunoglobulin can specifically bind.

As used herein, the term “epitope” or “antigenic determinant” or “idiotypic determinant” refers to a site on an antigen to which an immunoglobulin (or antigen binding fragment thereof) can specifically bind. Epitopes can be formed both from contiguous amino acids or noncontiguous amino acids juxtaposed by tertiary folding of a protein. Epitopes found on the Fab (variable) region of immunoglobulins are referred to as “idiotypic determinants” and comprise the immunoglobulin's “idiotype”. Epitopes formed from contiguous amino acids are typically retained on exposure to denaturing solvents, whereas epitopes formed by tertiary folding are typically lost on treatment with denaturing solvents. In the case of proteinaceous antigens, an epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, for example, Epitope Mapping Protocols in Methods in Molecular Biology, Vol. 66, G. E. Morris, Ed. (1996).

The term “domain” refers to a globular region of a heavy or light chain polypeptide comprising peptide loops (e.g., comprising 3 to 4 peptide loops) stabilized, for example, by beta-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. “Constant” domains on the light chain are referred to interchangeably as “light chain constant regions”, “light chain constant domains”, “CL” regions or “CL” domains). “Constant” domains on the heavy chain are referred to interchangeably as “heavy chain constant regions”, “heavy chain constant domains”, “CH” regions or “CH” domains). “Variable” domains on the light chain are referred to interchangeably as “light chain variable regions”, “light chain variable domains”, “VL” regions or “VL” domains). “Variable” domains on the heavy chain are referred to interchangeably as “heavy chain variable regions”, “heavy chain variable domains”, “VH” regions or “VH” domains).

The term “region” refers to a part or portion of an antibody chain or antibody chain domain (for example, a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein. As used herein, a “region” of an antibody is inclusive of regions existing in isolation (as antibody fragments) and as part of whole (intact) or complete antibodies. Thus, for example, an idiotype immunoglobulin comprising “at least an IgM constant region” encompasses embodiments in which the idiotype immunoglobulin is composed of only the constant region of the IgM (and, optionally, other non-IgM components), as well as embodiments in which the idiotype immunoglobulin is composed of more of the IgM than just the constant region (and, optionally, other non-IgM components).

As used herein, the terms “constant region” or “fragment crystallizable region” (Fc region) refers to that portion of the antibody (the tail region) that interacts with cell surface receptors called Fc receptors and some proteins of the complement system, and is composed of two heavy chains that contribute two or three constant domains depending on the class of the antibody (Janeway C A, Jr et al. (2001). Immunobiology. (5th ed.). Garland Publishing). In IgG, IgA and IgD antibody isotypes, the Fc region is composed of two identical protein fragments, derived from the second and third constant domains of the antibody's two heavy chains; IgM and IgE Fc regions contain three heavy chain constant domains (C_{H} domains 2-4) in each polypeptide chain. The Fc regions of IgGs hear a highly conserved N-glycosylation site (Janeway C A, Jr et al. (2001). Immunobiology. (5th ed.); Garland Publishing Rhoades R A, Pflanzer R G (2002). Human Physiology (4th ed.). Thomson Learning). The other part of an antibody, called the Fab region, contains variable sections that define the specific target that the antibody can bind. By contrast, the Fc region of all antibodies in a class are the same for each species; they are constant rather than variable. The terms “Fc region” and “Fab region” encompass these regions existing in isolation (as antibody fragments) and as part of a whole (intact) or complete, full-length antibody.

The terms “polynucleotide” and “nucleic acid molecule” are used interchangeably herein to refer to a polymeric form of nucleotides of any length, which contain deoxyribonucleotides, ribonucleotides, and analogs in any combination analogs. Polynucleotides may have any three-dimensional structure, and may perform any function, known or unknown. The term “nucleic acid molecule” includes double-, single-stranded, and triple-helical molecules. Unless otherwise specified or required, any embodiment of the invention described herein that is a nucleic acid molecule encompasses both the double-stranded form and each of two complementary single-stranded forms known or predicted to make up the double stranded form. In some embodiments, the nucleic acid molecule encodes an epitope or an antigen.

The following are non-limiting examples of nucleic acid molecules: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A nucleic acid molecule may be further modified after polymerization, such as by conjugation with a labeling component. Other types of modifications included in this definition are caps, substitution of one or more of the naturally occurring nucleotides with an analog, and introduction of means for attaching to proteins, metal ions, labeling components, other nucleic acid molecules, or a solid support.

The terms “polypeptide”, “peptide” and “protein” are used interchangeably herein to refer to polymers of amino acids of any length. The polymer may be linear or branched, it may comprise modified amino acids or amino acid analogs, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component.

The term “fusion polypeptide” refers to a polypeptide comprising regions in a different position in the sequence than occurs in nature. The regions may normally exist in separate proteins and are brought together in the fusion polypeptide; or they may normally exist in the same protein but are pieced in a new arrangement in the fusion polypeptide. Fusion polypeptides can be produced by linking two or more polypeptides together (for example, covalently), or by expressing nucleic acids encoding each fusion partner within a host cell, for example.

The term “sensitizing” in the context of immunity refers to inducing or increasing a humoral and/or cellular immune response against an epitope (such as a polypeptide) in the subject.

The term “tolerizing” in the context of immunity refers to reducing (eliminating or suppressing) an immune response against an epitope in the subject.

The term “adjuvant” refers to a substance incorporated into or administered simultaneously with an antigen which potentiates the immune response in response to that antigen but does not in itself confer immunity. A tetanus, diphtheria, and pertussis vaccine, for example, contains minute quantities of toxins produced by each of the target bacteria, but also contains some aluminum hydroxide. Aluminum salts are common adjuvants in vaccines sold in the United States and have been used in vaccines for over 70 years. The body's immune system develops an antitoxin to the bacteria's toxins, not to the aluminum, but would not respond enough without the help of the aluminum adjuvant. An adjuvant can also include cytokines such as granulocyte-monocyte colony stimulating factor (GM-CSF). In some cases, e.g., immunization of a subject against normally non-immunogenic tumor-derived idiotypes, foreign (non-self) carrier protein immunogens such as keyhole limpet hemocyanin (KLH), can also potentiate the immune response and serve as adjuvants.

### Determining Immunoglobulin Isotype (Class) of B-Cell Malignancies

Samples of malignant cells (e.g., tumor cells) can be obtained from a subject for isotyping by biopsy, fine-needle aspiration, or apheresis, for example. The immunoglobulin to be isotyped may be present on the malignant cell surface, within the malignant cell cytoplasm, or in the subject's blood. The method of collection will depend upon where the immunoglobulin-bearing cells or secreted immunoglobulin molecules are found. For example, depending upon the malignancy, samples can be obtained from lymph nodes, extra-nodal tissue, spleen, bone marrow, or blood (Alvarez-Vallina L. et al., Journal of Immunotherapy, 1995, 17:194-198).

Malignant cells can be isotyped by flow cytometry (Zabelegui N. et al., haeamatologica, 2004, 89(5):541-546). Antibodies specific for various isotypes are commercially available. For example, human anti-IgM antibodies are available from Miltenyi Biotec (Auburn Calif.). Other methods such as immunofluoroescence, immunohistochemistry of sections (e.g., from a biopsy), sequencing of the constant region on the heavy chain, immunoblot, etc. (Fakhrjou A. et al., Pakistan Journal of Biological Sciences, 2010, 13(4):194-197).

In some embodiments, the B-cell malignancy exhibits a predete mined immunoglobulin isotype or isotypes that is not an IgM isotype (a non-IgM immunoglobulin). In some embodiments, the B-cell the malignancy exhibits a predetermined immunoglobulin isotype or isotypes that is an IgM isotype (an IgM immunoglobulin). In some embodiments, the non-IgM immunoglobulin is IgG, IgA, IgD, IgE, or any combination of two or more of the foregoing (for example, IgM/IgA or IgM/IgG). In some embodiments, the non-IgM immunoglobulin is IgG1, IgG2, IgG3, IgG4, IgA1, IgA2, IgE, IgD, or any combination of the foregoing.

### Exemplary Disorders

Exemplary disorders which may be treated using the methods of the invention include B-cell malignancies and in particular, B-cell derived cancers or neoplasms such as, for example, non-Hodgkin's lymphoma, Hodgkin's lymphoma, chronic lymphocytic leukemia, mantle cell lymphoma and multiple myeloma. Additional B-cell derived cancers include, for example, B-cell prolymphocytic leukemia, lymphoplasmocytic leukemia, splenic marginal zone lymphoma, marginal zone lymphoma (extra-nodal and nodal), plasma cell neoplasms (e.g., plasma cell myeloma, plasmacytoma, monoclonal immunoglobulin deposition diseases, heavy chain diseases), and follicular lymphoma (e.g., Grades I, II, III, or IV).

In some embodiments, a malignancy treated using the methods of the present invention is a B-cell derived malignancy associated with the expression of one or more B-cell specific antigens such as, for example, CD3d, CD5, CD6, CD9, CD19, CD20, CD21, CD22, CD23, CD24, CD27, CD28, CD37, CD38, CD40, CD45, CD46, CD48, CD53, CD69, CD70, CD72, CD73, CD79a, CD79b, CD80, CD81, CD83, CD85a, CD85d, CD85e, CD85h, CD85i, CD85j, CD85k, CD86, CD96, CD98, CD100, CD121b, CD124, CD127, CD132, CD150, CD152, CD154, CD157, CD166, CD169, CD179a, CD179b, CD180, CD185, CD196, CD197, CD205, CDw210a, CD213a1, CD257, CD267, CD268, CD269, CD274, CD275, CD276, CD278, CD279, CD300a, CD300c, CD307, CD314, CD316, CD317, CD319, CD320, CDw327, and CD331. In a particular embodiment, a cancer treated using the methods of the invention is associated with the expression of CD-20. In another embodiment, a cancer treated using the methods of the invention is associated with the expression of CD-22. In yet another embodiment, a cancer treated using the methods of the invention is associated with the expression of both CD-20 and CD-22.

In some embodiments, a cancer treated using the methods of the invention is non-Hodgkin's lymphoma or NHL. Non-Hodgkin's lymphoma or NHL, is a cancer of the lymphoid tissue which is formed by several types of immune cells including B-cells and T-cells. About 85% of the non-Hodgkin's lymphomas are derived from B-cells. NHL is thought to occur when B-cells, which produce antibodies, begin to grow abnormally. In some embodiments, non-Hodgkin's lymphoma treated using the methods of the invention is associated with the expression of CD-20 on B-cells. In other embodiments, non-Hodgkin's lymphoma is associated with the expression of CD-22. In yet other embodiments, non-Hodgkin's lymphoma is associated with the expression of both CD-20 and CD-22.

In some embodiments, a cancer treated using the methods of the invention is Hodgkin's lymphoma, also referred to as Hodgkin's disease. The cancer cells in Hodgkin's disease are called Reed-Sternberg cells, after the two doctors who first described them in detail. Under a microscope they look different from cells of non-Hodgkin's lymphomas and other cancers, and are believed to be a type of malignant B lymphocyte.

In some embodiments, a cancer treated using the methods of the invention is chronic lymphocytic leukemia (CLL) which is derived from a small B lymphocyte. CLL is mostly found in the blood and in the bone marrow.

In further embodiments, a cancer treated using the methods of the invention is mantle cell lymphoma.

In some embodiments, the B-cell malignancy is multiple myeloma, associated with uncontrolled proliferation of antibody producing cells in the plasma, which develop from B-cells.

In some embodiments, the B-cell malignancy is non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, or Burkitt lymphoma/leukemia. In some embodiments, the B-cell malignancy is a mature B-cell lymphoma. In some embodiments, the mature B-cell lymphoma is B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, or Burkitt lymphoma/Burkitt cell leukemia.

In some embodiments, the mature B-cell lymphoma is a variant malignancy, for example, B-cell chronic lymphocytic leukemia/small lymphocytic lymphoma with monoclonal gammopathy/plasmacytoid differentiation, hairy cell leukemia variant, cutaneous follicle center lymphoma, diffuse follicle center lymphoma, blastoid mantle-cell lymphoma, morphologic variant of diffuse large B-cell lymphoma (for example, centroblastic, immunoblasti c, T-cell/histiocyte-rich, lymphomatoid granulomatosis type, anaplastic large B-cell, plasmablastic) or subtype of diffuse large B-cell lymphoma (for example, mediastinal (thymic) large B-cell lymphoma, primary effusion lymphoma, intravascular large B-cell lymphoma), morphologic variant of Burkitt lymphoma or Burkitt cell leukemia (for example, Burkitt-like lymphoma/leukemia, Burkitt lymphoma/Burkitt cell leukemia with plasmacytoid differentiation (AIDS-associated), or clinical or genetic subtype of Burkitt lymphoma/Burkitt cell leukemia (for example, endemic, sporadic, immunodeficiency-associated).

### Exemplary Autologous Idiotype Vaccines

In various embodiments of the methods of the present invention, an autologous idiotype vaccine is produced using a hybridoma technology. For example, a hybridoma cell-line may be developed which contains a tumor-specific antigen derived from a patient, which is unique to that patient and found exclusively on the surface of a B-lymphocyte associated with a B-cell derived cancer such as, for example, non-Hodgkin's lymphoma, Hodgkin's lymphoma, chronic lymphocytic leukemia, mantle cell lymphoma or multiple myeloma, and which is absent or expressed in decreased amounts in normal B-lymphocytes and other cells.

In some embodiments, an “autologous idiotype vaccine” includes an antigen associated with a B-cell derived cancer in a subject (for example, non-Hodgkin's lymphoma, Hodgkin's lymphoma, chronic lymphocytic leukemia, mantle cell lymphoma or multiple myeloma) linked to a carrier molecule, such as a carrier protein. Preferably, the carrier molecule is immunogenic, such as the immunogenic carrier protein KLH ((keyhole limpet hemocyanin) Kwak L W et al., N Engl. J. Med., 327:1209-1215 (1992); Hsu F J et al., Blood, 89:3129-3135 (1997); Schumacher K, J. Cancer Res. Clin, Oncol., 127(Suppl 2):R1-R2 (2001)). An exemplary autologous idiotype vaccine is BIOVAXID®.

In some embodiments, the autologous idiotype vaccine comprises an antigen associated with a B-cell derived malignancy in the subject, and wherein the antigen is produced by a hybridoma (see, for example, Lee S T et al., Expert Opin Biol Ther, 7(1):113-122 (2007); Flowers C R, Expert Rev Vaccines, 6(3):307-317 (2007); Neelapu S S and LW Kwak, Hematology, 243-249, (2007); Lee S-T. et al., Yonsei Medical Journal, 48(1):1-10 (2007); Ruffini P A et al., Haematologica, 87:989-1001 (2002), which are each incorporated herein by reference in their entirety). In some embodiments, the hybridoma is produced by fusion of a cancerous B-cell obtained from the subject and a murine/human heterohybridoma myeloma cell, such as the K6H6/B5 cell line. In some embodiments, the antigen-producing hybridoma is grown in a hollow-fiber bioreactor, such as those described in one or more of International Patent Publications WO 2007/139748 (Biovest International, Inc., filed May 21, 2007); WO 2007/139742 (Biovest International, Inc., filed May 21, 2007); WO 2007/139746 (Biovest International, Inc., filed May 21, 2007); WO 2007/136821 (Biovest International, Inc., filed May 21, 2007); and WO 2007/139747 (Biovest International, Inc., filed May 21, 2007), each of which are incorporated herein by reference in their entirety). The antigen can then be collected from the hollow-fiber bioreactor and purified (e.g., by affinity chromatography) prior to administration to the subject.

Preferably, in both the initial treatment with the autologous idiotype vaccine and in any (optional) booster doses of the autologous idiotype vaccine, the purified antigen is conjugated to a carrier molecule, such as an immunogenic earlier protein (e.g., KLH), prior to administration to the subject.

In some embodiments, the autologous idiotype vaccine comprises a chimeric idiotype immunoglobulin comprising at least an IgM constant region, and a variable region derived from a non-IgM immunoglobulin expressed by the malignancy. In some embodiments, the autologous idiotype vaccine comprises a chimeric idiotype immunoglobulin comprising at least an IgM constant region, and a variable region derived from an IgM immunoglobulin expressed by the malignancy. The chimeric idiotype immunoglobulin can be produced recombinantly by introducing a genetic construct into a host cell, wherein the genetic construct comprises a nucleic acid sequence encoding the IgM constant region and a nucleic acid sequence encoding the variable region of the immunoglobulin expressed by the malignant cell, wherein the isotype of the immunoglobulin is not IgM (the non-IgM variable region), and wherein the nucleic sequences are expressed by the host cell.

The type of host cell used to produce the chimeric idiotype immunoglobulin may be any capable of expressing the nucleic acids encoding the IgM constant region and/or variable region of the immunoglobulin expressed by the malignant cell. For example, the host cell may be a mammalian cell, insect cell, bacterial cell, plant cell, viral cell, or fungal cell (see, for example, Bendandi, M. et al., “Rapid, high-yield production in plants of individualized idiotype vaccines for non-Hodgkin's lymphoma,” Ann Oncol., 21(12):2420-2427 (2010); Bertinetti, C. et al., “Cloning of idiotype immunoglobulin genes in B cell lymphomas by anchored PCR and production of individual recombinant idiotype vaccines in Escherichia coli,” Eur J Haematol, 77(5):395-402 (2006); Tchoudakova, A. et al., “High level expression of functional human IgMs in human PER.C6 cells,” MAbs, (2):163-71 (2009); Wood, C. R. et al., “High level synthesis of immunoglobulins in Chinese hamster ovary cells,” J Immunol, 145(9): p. 3011-6 (1990)). Host cells useful for expression of polynucleotides encoding the immunoglobulin domains may be primary cells or cells of cell lines. The host cells may be tumor cells (transformed cells) or non-tumor cells. Mammalian cell lines available as hosts for expression are known in the art and are available from depositories such as the American Type Culture Collection. These include but are not limited to HeLa cells, human embryonic kidney (HEK) cells, Chinese hamster ovary (CHO) cells, baby hamster kidney (BHK) cells, and others.

Both prokaryotic and eukaryotic host cells may be used for expression of desired coding sequences when appropriate control sequences (e.g., promoter sequences) that are compatible with the designated host are used. For example, among prokaryotic hosts, Escherichia coli may be used. Also, for example, expression control sequences for prokaryotes include but are not limited to promoters, optionally containing operator portions, and ribosome binding sites. Eukaryotic hosts include yeast, insect, and mammalian cells in culture systems. Pichia pastoris, Saccharomyces cerevisiae and S. carlsbergensis are commonly used yeast hosts.

As indicated above, the type of host cell used may be, for example, a mammalian cell, insect cell, bacterial cell, plant cell, viral cell, or fungal cell. Trichoplusia ni and Spodoptera frugiperda (Sf9) are examples of insect cells that may be used. The baculovirus expression system is an attractive alternative to antibody production in E. coli and mammalian cells, for example. The baculovirus/insect cell system also circumvents solubility problems that may be encountered when recombinant proteins are overexpressed in prokaryotes. In addition, insect cells contain the eukaryotic post-translational modification machinery responsible for correct folding, disulfide formation, glycosylation, P-hydroxylation, fatty acid acylation, prenylation, phosphorylation and amidation not present in prokaryotes.

### Exemplary Antibodies for Combination or Adjunctive Treatment

In various methods of the present invention, malignancies derived from B-cells can be treated using a combination of an autologous idiotype vaccine with one or more other therapies, such as a monoclonal antibody. The combination therapy may be consecutive (e.g., antibody therapy followed by autologous idiotype vaccine therapy) or contemporaneous. In some embodiments, malignancies derived from B-cells can be treated using a combination of an autologous idiotype vaccine with a monoclonal antibody which selectively binds a B-cell specific antigen. Examples of monoclonal antibody therapies include rituximab, tositumomab, ibritumomab tiuxetan, epratuzumab alemtuzumab, (see, for example, Cheson B. D. and J. P. Leonard, N. Engl. J. Med., 359(6):613-626 (2008)). Preferably, in any subjects receiving any of the pan-B-cell immunoablative therapies (e.g., Rituxan, Bexxar, Zevalin), any booster administrations of the autologous idiotype vaccine are administered at least about one month after such immunoablative therapies, as it typically takes approximately 14-21 days for B-cell recovery.

In some embodiments of the present invention, an antibody is a monoclonal antibody that specifically binds CD-20 on a B-cell. In other embodiments, an antibody is a monoclonal antibody that specifically binds CD-22 on a B-cell. However, without wishing to be bound by theory, it is contemplated that a human or humanized monoclonal antibody that selectively binds any one of B-cell specific antigens CD3d, CD5, CD6, CD9, CD19, CD20, CD21, CD22, CD23, CD24, CD27, CD28, CD37, CD38, CD40, CD45, CD46, CD48, CD52, CD53, CD69, CD70, CD72, CD73, CD74, CD79a, CD79b, CD80, CD81, CD83, CD85a, CD85d, CD85e, CD85h, CD85i, CD85j, CD85k, CD86, CD96, CD98, CD100, CD121b, CD124, CD127, CD132, CD150, CD152, CD154, CD157, CD166, CD169, CD179a, CD179b, CD180, CD185, CD196, CD197, CD205, CDw210a, CD213a1, CD257, CD267, CD268, CD269, CD274, CD275, CD276, CD278, CD279, CD300a, CD300c, CD307, CD314, CD316, CD317, CD319, CD320, CDw327, CD331, Death receptor, or HLA-DR may be used in the methods of the invention.

Commercially available monoclonal antibodies that specifically bind B-cell specific antigens include, for example, rituximab, which binds CD-20, and epratuzumab, which binds CD-22 (see, for example, Cheson B. D. and J. P. Leonard, N. Engl. J. Med., 359(6):613-626 (2008)).

Antibodies or antigen-binding portions thereof can be tested for binding to a B-cell or a B-cell specific antigen by, for example, standard assays known in the art, such as ELISA, FACS analysis and/or Biacore analysis.

Antibodies or antigen-binding portions useful in the methods of the invention may be labeled with a detectable substance using well known techniques. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹⁴C, ¹²³I, ¹²⁴I, ¹²⁵I, ¹³¹I, ^{99m}Tc, ³⁵S or ³H.

### Modes of Administration

The various compounds used in the methods described herein may be administered orally, parenterally (e.g., intravenously), intramuscularly, sublingually, buccally, rectally, intranasally, intrabronchially, intrapulmonarily, intraperitoneally, topically, transdermally and subcutaneously, for example. The amount of compound administered in a single dose may dependent on the subject being treated, the subject's weight, the manner of administration and the judgment of the prescribing physician. Generally, however, administration and dosage and the duration of time for which a composition is administered will approximate that which are necessary to achieve a desired result.

In general, a therapeutically effective amount of a monoclonal antibody such as, for example, an antibody that specifically binds CD-20 or CD-22, from about 0.0001 mg/Kg to 0.001 mg/Kg; 0.001 mg/kg to about 10 mg/kg body weight or from about 0.02 mg/kg to about 5 mg/kg body weight. In some embodiments, a therapeutically effective amount of a monoclonal antibody is from about 0.001 mg to about 0.01 mg, about 0.01 mg to about 100 mg, or from about 100 mg to about 1000 mg, for example.

In some embodiments, a therapeutically effective amount of an autologous idiotype vaccine is from about 0.001 mg to about 0.01 mg, about 0.01 mg to about 100 mg, or from about 100 mg to about 1000 mg, for example. In some embodiments, an effective amount of the autologous idiotype vaccine is one or more doses of 0.5 mg.

In some embodiments, an effective amount of an antibody administered to a subject having non-Hodgkin's lymphoma, Hodgkin's lymphoma, chronic lymphocytic leukemia or multiple myeloma between about 100 mg/m² and 200 mg/m², or between about 200 mg/m² and 300 mg/m² or between about 300 mg/m² and 400 mg/m². In a particular embodiment, an effective amount of a monoclonal antibody that selectively binds a B-cell specific antigen is about 375 mg/m².

The optimal pharmaceutical formulations for a desired monoclonal antibody can be readily determined by one or ordinary skilled in the art depending upon the route of administration and desired dosage. (See, for example, Remington's Pharmaceutical Sciences, 18th Ed. (1990), Mack Publishing Co., Easton, Pa., the entire disclosure of which is hereby incorporated by reference).

Antibodies for use in the methods or compositions described herein can be formulated for the most effective route of administration, including for example, oral, transdermal, sublingual, buccal, parenteral, rectal, intranasal, intrabronchial or intrapulmonary administration.

In some embodiments, the vaccine compositions used in the methods of the present invention include one or more cytokines such as, for example, GM-CSF. GM-CSF is a potent immunostimulatory cytokine with efficacy in promoting anti-tumor response, particularly T cell responses. In general, however, any cytokine or chemokine that induces inflammatory responses, recruits antigen presenting cells (APC) to the tumor and, possibly, promotes targeting of antigen presenting cells (APC) may be used in the vaccine compositions.

The autologous idiotype vaccines useful in the methods of the present invention may be administered by any conventional route including oral and parenteral. Examples of parenteral routes are subcutaneous, intradermal, transcutaneous, intravenous, intramuscular, intraorbital, intracapsular, intrathecal, intraspinal, intracisternal, intraperitoneal, etc. Preferably, the primary treatment and one or more booster doses of the autologous idiotype vaccine are administered by the same route, e.g., subcutaneously.

An effective amount of a vaccine composition administered to a subject will vary from individual to individual and can be, for example, between about 0.01 μg/kg and about 1 mg/kg body weight. The amount of the immunogen per dose can range from about 0.01 mg to 100 mg of protein per subject per injection.

Administration of the immunogenic (vaccine) composition is preferably by injection on one or multiple occasions to produce systemic immunity. In general, multiple administrations of the vaccine in a standard immunization protocol are used, as is standard in the art. For example, the vaccines can be administered at approximately two to six week intervals, or monthly, for a period of from one to six inoculations in order to provide protection. The vaccine may be administered by any conventional route including oral and parenteral. Examples of parenteral routes are subcutaneous, intradermal, transcutaneous, intravenous, intramuscular, intraorbital, intracapsular, intrathecal, intraspinal, intracisternal, intraperitoneal, etc.

Without wishing to be bound by theory, it is contemplated that vaccination may result in a systemic immune response, which includes either or both of an antibody response and a cell-mediated immune response, which will provide an anti-cancer therapeutic effect and/or result in antibodies and activated. T lymphocytes of various classes which may be used themselves as therapeutic agents, for example, for producing passive immunity in cancer-bearing subjects.

The vaccine compositions used in the methods of the present invention may further include one or more adjuvants or immunostimulatory agents. Examples of adjuvants and immunostimulatory agents include, but are not limited to, aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives, liposomes, levamisole, DEAE-dextran, blocked copolymers or other synthetic adjuvants. Such adjuvants are readily commercially available.

Depending on the intended mode of administration, the compounds used in the methods described herein (e.g., autologous idiotype vaccines) may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. Each dose may include an effective amount of a compound used in the methods described herein in combination with a pharmaceutically acceptable carrier and, in addition, may include other medicinal agents, pharmaceutical agents, carriers, adjuvants, diluents, etc.

Liquid pharmaceutically administrable compositions can prepared, for example, by dissolving, dispersing, etc., a compound for use in the methods described herein and optional pharmaceutical adjuvants in an excipient, such as, for example, water, saline aqueous dextrose, glycerol, ethanol, and the like, to thereby form a solution or suspension. For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; see, for example, Remington's Pharmaceutical Sciences, 18th Ed. (1990), Mack Publishing Co., Easton, Pa., the entire disclosure of which is hereby incorporated by reference).

### Methods of Treatment

Methods of treatment described herein encompass methods of eliminating or substantially reducing a B-cell derived malignancy such as, for example, non-Hodgkin's lymphoma, Hodgkin's lymphoma, chronic lymphocytic leukemia, mantle cell lymphoma and multiple myeloma.

In some embodiments, the B-cell derived malignancy to be treated is selected from among non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, and Burkitt lymphoma/leukemia.

A subject having non-Hodgkin's lymphoma, Hodgkin's lymphoma, chronic lymphocytic leukemia, mantle cell lymphoma or multiple myeloma can be diagnosed using standard techniques known in the art. For example, a diagnosis may be made by removing a part of a lymph node and examining the cells under a microscope. Biopsies may also be taken from other body tissues.

Subsequent to diagnosis, a subject having non-Hodgkin's lymphoma, Hodgkin's lymphoma, chronic lymphocytic leukemia, mantle cell lymphoma or multiple myeloma can be treated using methods of the invention.

In some embodiments, a subject having non-Hodgkin's lymphoma or Hodgkin's lymphoma or chronic lymphocytic leukemia, mantle cell lymphoma or multiple myeloma is administered an effective amount of an autologous idiotype vaccine, which may optionally be administered in conjunction with an effective amount of GM-CSF, followed by re-administration of the autologous anti-idiotype vaccine one or more times as a booster.

In some embodiments, a subject having non-Hodgkin's lymphoma or Hodgkin's lymphoma or chronic lymphocytic leukemia or mantle cell lymphoma or multiple myeloma is administered an autologous idiotype vaccine (optionally in conjunction with GM-CSF) and an effective amount of a monoclonal antibody which specifically binds a B-cell specific antigen, e.g., CD-20 or CD-22, followed by re-administration of the autologous anti-idiotype vaccine, without the monoclonal antibody, as a booster.

In some embodiments, the booster dose(s) of the autologous idiotype vaccine is administered at least about 20 months after the initial treatment (i.e., at least 20 months after last vaccination). In some embodiments, the booster dose(s) of the autologous idiotype vaccine is administered to the subject about 24 months to about 30 months after completion of the initial treatment (i.e., after last vaccination). In some embodiments, the booster doses of the autologous idiotype vaccine are administered to the subject about 24 months to about 30 months after completion of the initial treatment and administered again in about 12 to about 18 months thereafter. In some embodiments, the booster doses of the autologous idiotype vaccine are administered to the subject about 24 months to about 30 months after completion of the initial treatment and administered again in about 12 to about 18 months thereafter, and periodically at about every 12 to 18 months thereafter.

The initial treatment with the autologous idiotype vaccine can comprise one or more administrations. Preferably, the initial treatment is a regimen comprising a plurality of administrations of the autologous idiotype vaccine. In some embodiments, the initial treatment comprises five administrations of the autologous idiotype vaccine over a period of about 6 months. In some embodiments, the autologous idiotype vaccine comprises an antigen associated with a B-cell derived malignancy in the subject, and a carrier molecule linked to the antigen, and the initial treatment comprises administration (e.g., subcutaneous) of 0.01 mg to about 100 mg of the autologous idiotype vaccine (day 1) and about 50 μg/m²/day to about 200 μg/m²/day granulocyte monocyte-colony stimulating factor (days 1-4) at about 1, 2, 3, 4, and 6 months. In some embodiments, the autologous idiotype vaccine comprises an antigen associated with a B-cell derived malignancy in the subject, and keyhole limpet hemocyanin linked to the antigen, and the initial treatment comprises administration (e.g., subcutaneous) of 0.5 mg of the autologous idiotype vaccine (day 1) and 100 μg/m²/day granulocyte monocyte-colony stimulating factor (days 1-4) at about 1, 2, 3, 4, and 6 months.

In some embodiments, the booster dose comprises about 0.01 mg to about 100 mg autologous idiotype vaccine per administration (e.g., subcutaneous). In some embodiments, the booster dose comprises about 0.5 mg autologous idiotype vaccine per administration (e.g., subcutaneous).

In some embodiments, the subject has undergone a different therapy (i.e., other than the autologous idiotype vaccine therapy) prior to the initial treatment, such as chemotherapy and/or immunotherapy. In some embodiments, the different therapy comprises therapy with a monoclonal antibody, such as rituximab, tositumomab, ibritumomab tiuxetan, or epratuzumab (see, for example, Cheson B. D. and J. P. Leonard, N. Engl. J. Med., 359(6):613-626 (2008)). In some embodiments, the different therapy comprises a radioimmunotherapy, such as ibritumomab tiuxetan. In some embodiments, the different therapy comprises a regimen of PACE (prednisone, doxorubicin, cyclophosphamide, and etoposide) or CHOP-R (cyclophosphamide, hydroxydaunrubicin, oncovin, prednisone/prednisolone, and rituximab). Preferably, the different therapy induces complete remission in the subject prior to the initial treatment with the vaccine. Preferably, the subject is in complete remission at the time of the initial treatment with the vaccine. Preferably, the subject is in complete remission at the time that each of the one or more booster doses is administered.

Endogenous mechanisms for controlling autoimmune responses (natural tolerance) and of inducing tolerance (adaptive tolerance) exist. T-regulatory lymphocytes (T-regulatory cells or T-regs) are a specialized subset of CD4+ T cells implicated in the suppression of immune response, fulfilling an important role in the maintenance of immune homeostasis (Sakaguchi S. “Regulatory T cells: key controllers of immunologic self-tolerance,” Cell, 101:455-458 (2000)). T-regs differ from other CD4⁺ cells in expressing high levels of CD25 and by expression of the forkhead/winged helix transcription factor (Foxp3). Under some circumstances, it may be desirable to inhibit T-reg cell activity and/or reduce the number of T-regs in a subject (i.e., to inhibit the immunosuppressive effects of T-regs) prior to vaccinating the subject with an autologous idiotype vaccine. Accordingly, in some embodiments of the invention, the subject has reduced T-regulatory cell activity and/or reduced numbers of T-regulatory cells at the time of administration of an idiotype vaccine. Reduced T-regulatory cell activity and/or reduced T-regulatory cell numbers may be achieved in a subject by administering an inhibitor of T-regulatory cells to the subject. The reduced T-regulatory cell activity and/or reduced numbers of T-regulatory cells can be relative to the normal activity and/or cell numbers in the subject and/or relative to a normal control population, for example. The normal T-reg level may be one which is consistent with an immunosuppressive state in the subject. As used herein, the teiin “T-reg level” refers to T-reg cell activity, T-reg cell number, or both.

Agents capable of inhibiting T-reg immunosuppressive activity and/or Treg numbers, and which may be utilized in the invention, are known (Cohen A. D. et al., “Agonist anti-GITR antibody enhances vaccine-induced CD8(+) T-cell responses and tumor immunity”, Cancer Res 66:4904-49-12 (2006); Onizuka S. et al., “Tumor rejection by in vivo administration of anti-CD25 (interleukin-2 receptor alpha) monoclonal antibody” Cancer Res, 59:3128-3133 (1999); Shimizu J. et al., “Induction of tumor immunity by removing CD25+CD4+ T cells: a common basis between tumor immunity and autoimmunity,” J. Immunol., 163:5211-5218 (1999); Tanaka H. et al., “Depletion of CD4+ CD25+ regulatory cells augments the generation of specific immune T cells in tumor-draining lymph nodes,” J. Immunother, 25:207-217 (2002); Ko K. et al., “Treatment of advanced tumors with agonistic anti-GITR mAB and its effects on tumor-infiltrating Foxp3+CD25+CD4+ regulatory T cells,” J. Exp. Med., 202:885-891 (2005); Ghiringhelli F. et al., “CD4+CD25+ regulatory T cells suppress tumor immunity but are sensitive to cyclophosphamide which allows immunotherapy of established tumors to be curative,” Eur. J. Immunol., 34:336-344 (2004); Galustian C. et al., “The anti-cancer agents lenalidomide and pomalidomide inhibit proliferation and function of T regulatory cells” Cancer Immunol Immunother., 58(7):1033-1045 (2009); Houot R. et al., “T-cell modulation combined with intratumoral CpG cures lymphoma in a mouse model without the need for chemotherapy”, Blood, 113(15):3546-3552 (2009); Nizar S. et al., “T-regulatory cell modulation: the futre of cancer immunotherapy?”, British Journal of Cancer, 100:1697-1703; and Dias de Rezende, L. C. et al., “Regulatory T cell as a target for cancer therapy”, Arch. Immunol. Ther. Exp., 58:179-190 (2010)).

Examples of Treg inhibitors include, but are not limited to, lenalidomide, pomalidomide, oxazaphosphorines such as cyclophosphamide, anti-CD25 monoclonal antibody, IL-2Ra monoclonal antibody, and anti-glucocorticoid-induced tumor necrosis factor receptor (anti-GITR) monoclonal antibody. In some embodiments, the inhibitor of T-regulatory cells reduces the activity and/or reduces the number of CD4+ CD25_{Hi}FoxP3⁺ natural T-regulatory cells in the subject. In some embodiments, the methods of the invention comprise administering a T-regulatory cell inhibitor to the subject, and subsequently administering an idiotype vaccine to the subject (e.g., an idiotype vaccine comprising an autologous idiotype immunoglobulin comprising at least an IgM constant region).

Another aspect of the invention features a method for selecting a treatment for a B-cell malignancy, comprising determining the T-regulatory (T-reg) cell level (T-reg cell number and/or T-reg activity) in the subject; wherein if the T-reg cell level is consistent with a normal T-reg cell level, an effective amount of a T-reg cell inhibitor is administered to the subject prior to administration of a vaccine of the invention. The T-reg cell level can be determined by obtaining one or more biological samples from the subject (for example, blood, peripheral blood, synovial fluid, or other biological tissue or fluid that may be sampled and in which T-reg cells are found) and determining the T-reg cell level in the sample(s) prior to administration of a vaccine of the invention. Ideally, the immunosuppressive effect of T-reg cells in the subject is inhibited or reduced to maximize the clinical effectiveness of the subsequently administered vaccine. Thus, preferably, the T-reg cell inhibitor is administered to the subject until the T-reg cell level in the subject is below that of a threshold, immunosuppressive T-reg cell level. In some embodiments, the T-reg cell level is determined two or more times and the T-reg cell inhibitor is administered to the subject until the T-reg cell level in the subject is below that of a threshold, immunosuppressive T-reg cell level, prior to administration of the vaccine. T-reg cell level can be determined by methods known in the art. For example, T-reg cells in a sample can be quantified by flow cytometry. Sub-populations of T-reg cells can be targeted for level determination, such as CD4+ CD25HIFoxp3+ cells.

In methods of the invention, determining T-reg cell level in a subject may involve comparing the observed level to that of a reference T-reg cell level or suitable control (for example, to assess whether T-reg cell level is below, equal to, or above a threshold level, e.g., a “normal” level). A “suitable control” is a predetermined value associated with T-reg cell level useful for comparison purposes, which can take many different forms. Exemplary forms include, but are not limited to, for example, T-reg cell numbers, a transcription rate, mRNA level, translation rate, protein level, protein structure, biological activity, cellular characteristic or property, genotype, phenotype, enzymatic activity etc. associated with T-reg cells. In some embodiments, a “suitable control” is a predetermined T-reg cell activity, which is compared to T-reg cell activity in a sample obtained from a subject being identified as suitable or not suitable for treatment with a vaccine of the invention. In other embodiments, a “suitable control” is a predetermined T-reg cell number, which is compared to T-reg cell number in a sample obtained from a subject being identified as suitable or not suitable for treatment with a vaccine of the invention. In other embodiments, a “suitable control” is a predetermined T-reg cell number and activity, which is compared to T-reg cell number and activity in a sample obtained from a subject being identified as suitable or not suitable for treatment with a vaccine of the invention. In other embodiments, a “suitable control” is a predetermined T-reg cell level, which is compared to a T-reg cell level in a sample obtained from a subject in which a clinical measure was achieved, for example an T-reg cell level obtained from cells in a subject who reached or failed to reach a desired immune response.

In some embodiments, a “suitable control” can be a single cut-off value, such as a median or mean. A single cut-off value can be established, for example, based upon comparative groups, such as in groups having a T-reg level which reduces a desirable immune response to a vaccine of the invention and/or which interferes or impedes a desired clinical outcome following treatment with a vaccine of the invention. For example, samples can be derived from various individuals or blood banks and a T-reg cell level can be measured in each sample prior to being subjected to treatment with a vaccine of the invention. Consequently, a single cut-off value can be based on the mean of T-reg cell number and/or activity in samples which are immunesuppressive to an extent that reduces a desirable immune response to a vaccine of the invention and/or which interferes or impedes a desired clinical outcome following treatment with a vaccine of the invention. Another comparative group can be, for example, a T-reg cell level in a group of individuals with a family history of successful treatment with a vaccine of the invention and a group without such a family history. Another comparative group can be, for example, a T-reg cell level in a group of individuals with a history of treatment with a vaccine of the invention having achieved maximal immune response and/or clinical outcome and a group having not achieved maximal immune response and/or clinical outcome.

In some embodiments of the methods of the present invention, a subject is identified as being suitable for vaccine treatment if the T-reg cell level measured in a sample (for example, blood sample) obtained from the subject is consistent with an “suitable control.” By “consistent with a suitable control,” is meant that the T-reg cell level is either equal to or below a predetermined T-reg cell level control, in case of a single cut-off value, or the T-reg cell level falls within a range for a predetermined T-reg cell level control. In some embodiments, a subject is identified as being suitable for vaccine treatment if the T-reg cell level in a sample from the subject is consistent with a maximal immune response (non-immune suppressed). By “consistent with a maximal immune response,” is meant that the T-reg cell level is either equal to or lower than a predetermined “immunosuppressive level,” in case of a single cut-off value, or the T-reg cell level falls within a range for a predetermined immunsuppressive level. In this way, it can be determined whether a subject is suitable for vaccine treatment (e.g., the T-reg cell level in a sample from the subject is consistent with a maximal immune response or “non-immune suppressed) or whether the subject should be administered a T-reg cell inhibitor (e.g., the T-reg cell level in a sample from the subject is inconsistent with or below a maximal immune response or “immune suppressed”).

Another aspect of the invention features a kit for treatment of a B-cell malignancy, comprising at least one autologous idiotype vaccine and printed instructions for using the vaccine for treatment of the B-cell malignancy. In some embodiments, the kit further comprises an immune adjuvant and/or one or more reagents for assessing immune response in a subject. In some embodiments, the idiotype vaccine comprises an autologous idiotype immunoglobulin linked to a carrier molecule.

Another aspect of the invention features a kit for assessing a humoral response to a vaccine of the invention, comprising an assay for detection of anti-idiotype immunoglobulins in a sample through their capacity to bind to the vaccine idiotype; and printed instructions for using the assay to detect the humoral response. The assay may be an enzyme-linked immunsorbent assay (ELISA), for example. The assay can be a colorimetric, chemiluminescent, fluorescent, or radioactive assay, for example.

Another aspect of the invention features a kit for assessing a cellular response to a vaccine of the invention, comprising an assay for detection of one or more activation markers, cytokines, growth factors, or cell subsets indicative of a cellular response, or a combination of two or more of the foregoing. The assay may be an enzyme-linked immunsorbent assay (ELISA), for example. The assay can be a colorimetric, chemiluminescent, fluorescent, or radioactive assay, for example.

Another aspect of the invention features a kit for detecting the T-regulatory (T-reg) cell response before, during, and after administration of a T-reg cell inhibitor prior to administration of a vaccine of the invention, wherein the kit comprises one or more reagents for assessing T-reg cell response in a subject; and printed instructions for making the assessment. In some embodiments, the kit further comprises a T-reg cell inhibitor.

In the various kits of the invention, each kit can include instructions or packaging materials that describe how to use a compound or composition (e.g., a reagent) of the kit. Each kit can include one or more containers for each component of the kit. Containers of the kits can be of any suitable material, e.g., glass, plastic, metal, etc., and of any suitable size, shape, or configuration.

### Assessing Immune Response

The methods of the invention may further comprise assessing whether an immune response to the autologous idiotype vaccine has been elicited in the subject and, optionally, determining whether the immune response against the vaccine has subsequently diminished (e.g., in character and/or extent). Optionally, the methods can include administering at least one booster dose of the autologous idiotype vaccine to the subject if the immune response against the vaccine is determined to have diminished.

An assessment can be made of the nature and/or extent of the subject's immune response to the vaccine (e.g., cellular and/or humoral response) one or more times after the initial treatment with the vaccine. Preferably, an assessment of the subject's immune response is also made before the subject's initial treatment with the autologous anti-idiotype vaccine (e.g., to establish a control or base-line for comparison to a subsequent assessment or assessments post-treatment). The subject's immune response to the vaccine can also be monitored by making an assessment before and after each booster dose is given. The timing and frequency of booster doses can be at the physician's discretion, and/or can be dependent on the results of assessments of the subject's immune response to the vaccine. For example, if the immune response is considered to be diminished (e.g., reduced or impaired in character and/or extent) following one of these assessments (e.g., either through loss of antibody response and/or a reduction of tumor-reactive T-cells or cytokines), it would indicate that the subject lost some of the immune response against the B-cell idiotype and therefore lost some anti-tumor immunity induced by the first cycle of vaccination. The physician could therefore consider administering a booster dose (e.g., one or more booster injections) or series of booster doses to the subject.

When assessing the subject's immune response, the immune response against the B-cell idiotype is preferably assessed. However, the assessment can include an assessment of the subject's immune response against any component of the vaccine. For example, an assessment of the subject's immune response against the anti-idiotype, or against a carrier molecule (e.g., KLH), or against both, can be made.

In some embodiments, enzyme-linked immunosorbent assays (ELISA) and/or T-cell proliferation assays are performed for detection of anti-Id humoral and/or cellular responses after vaccination (Hsu F. J. et al., “Tumor-specific idiotype vaccines in the treatment of patients with B-cell lymphoma—long term results of a clinical study,” Blood, 1997, 89:3129-3135).

The subject's immune response can be monitored by making multiple assessments after the initial treatment at uniform time intervals (e.g., every three months, every six months, every nine months, or annually) or at non-uniform time intervals. Monitoring of the subject's immune response to the vaccine can continue for a pre-determined period of time, for a time determined based on therapeutic outcome, or indefinitely. Preferably, the subject's immune response is monitored from a time period starting prior to initial vaccination and continuing for a period of at least five years, or indefinitely.

Typically, each assessment will involve obtaining an appropriate biological sample from the subject. The appropriate biological sample will depend upon the particular aspect of the subject's immune response to be assessed (e.g., depending upon the particular assay). For example, in some embodiments, the biological sample will be one or more specimens selected from among blood, peripheral blood mononuclear cells (PBMC), and B-cell derived tumor. Samples for assessments are taken at a time point appropriate to obtain information regarding the immune response at the time of interest. For example, a sample may be taken from the subject from a time prior to vaccination and additional samples may be taken from the subject periodically after vaccination to determine the nature and extent of the immune responses observed.

In some embodiments, assessment of the immune response includes assessment of one or more of the following aspects of the immune response: anti-idiotype (anti-Id) humoral responses; B-cell derived tumor-specific antibodies; tumor-reactive T-cell precursor frequencies (e.g., via an IFN-gamma response); biomarkers in the B-cell derived tumor that correlate with clinical outcome following autologous anti-idiotype vaccine therapy; and B-cell derived tumor-specific CD4+ and CD8+ T-cell responses.

Preferably, the immune response is assessed by conducting one or more humoral response assays and/or cellular response assays, such as those described by Neelapu et al, (Nature Medicine, 11(9):986-991 (2005)), which is incorporated herein by reference in its entirety. Peripheral blood B and T cells can be collected from the subject and blood counts can be determined, including but not limited to CD3-CD19+ B cells, CD3+CD4+ T cells, and CD3+CD8+ T cells. Tumor cells can be determined, and PBMCs isolated. Both B-cells and tumor cells can be activated with recombinant CD40 ligand trimer, as described in Neelapu et al. (2005). Depending on the type of immune response to be assessed (e.g., humoral, cellular, or both), one or more of the following assays may be used:
Humoral immune response assay: to assess anti-Id humoral responses and tumor-specific antibodies (see, for example, Kwak et al., Lancet, 345:1016-1020 (1995), which is incorporated herein by reference in its entirety).
IFN-gamma ELISPOT assay: to assess tumor-reactive T-cell precursor frequencies via an IFN-gamma response (see, for example, Malyguine et al., J. Trans. Med., 2:9 (2004) and Neelapu et al., Clin. Cancer Res., 10:8309-8317 (2004), which are each incorporated herein by reference in its entirety).
Cytokine induction assay: to assess biomarkers in the tumor that correlate with clinical outcome following autologous anti-idiotype vaccine therapy (see, for example, Neelapu et al. (2004)).
Intracellular cytokine assay: to assess tumor-specific CD4+ and CD8+ T-cell responses (Neealapu et al., J. Cancer Res. Clin. Oncol., 127 Suppl. 2, R14-19 (2001)).

Assays such as those listed above (either individually or in combination) can be used to periodically monitor (e.g., every 3, 6 months to 1 year) after a patient receives a course of the autologous idiotype vaccine, and may be used to determine an optimal schedule of booster vaccinations. In that case, if the immune response is considered to be reduced or impaired following one of these periodic tests (e.g., either through loss of antibody response and/or a reduction of tumor-reactive T-cells or cytokines), then the subject would be considered to have lost some of the anti-tumor immunity induced by the first cycle of vaccination. The physician could therefore consider administering a booster injection or series of injections to the subject.

### Exemplified Embodiments

Following are exemplified embodiments of the invention.

### Embodiment 1

A method for preparing an autologous idiotype vaccine for treatment of a B-cell malignancy in a subject in which the immunoglobulin isotype or isotypes exhibited by the malignancy has been predetermined, said method comprising preparing an autologous idiotype vaccine for the subject, wherein the vaccine comprises an idiotype immunoglobulin comprising at least an IgM constant region.

### Embodiment 2

A method for treating a B-cell malignancy in a subject in which the immunoglobulin isotype or isotypes exhibited by the malignancy have been predetermined, comprising administering an autologous idiotype vaccine to the subject, wherein the vaccine comprises an autologous idiotype immunoglobulin comprising at least an IgM constant region.

### Embodiment 3

The method of embodiment 1 or 2, wherein the malignancy exhibits a predetermined immunoglobulin isotype or isotypes that is not an IgM isotype (a non-IgM immunoglobulin).

Embodiment 4: The method of embodiment 1 or 2, wherein the malignancy exhibits a predetermined immunoglobulin isotype or isotypes that is an IgM isotype (an IgM immunoglobulin).

### Embodiment 5

The method of embodiment 3, wherein the non-IgM immunoglobulin is IgG, IgA, IgD, IgE, or any combination of two or more of the foregoing (for example, IgM/IgA or IgM/IgG).

### Embodiment 6

The method of embodiment 5, wherein the non-IgM immunoglobulin is IgG1, IgG2, IgG3, IgG4, IgA1, IgA2, IgE, IgD, or any combination of the foregoing.

### Embodiment 7

The method of embodiment 3 or 4, wherein the vaccine comprises a chimeric idiotype immunoglobulin comprising at least an IgM constant region, and a variable region derived from a non-IgM immunoglobulin expressed by the malignancy.

### Embodiment 8

The method of embodiment 3 or 4, wherein the vaccine comprises a chimeric idiotype immunoglobulin comprising at least an IgM constant region, and a variable region derived from an IgM immunoglobulin expressed by the malignancy.

### Embodiment 9

The method of embodiment 6 or 7, wherein the chimeric idiotype immunoglobulin is produced recombinantly by introducing a genetic construct into a host cell, wherein the genetic construct comprises a nucleic acid sequence encoding the IgM constant region and a nucleic acid sequence encoding the variable region of the immunoglobulin expressed by the malignant cell wherein the isotype of the immunoglobulin is not IgM, and wherein the nucleic sequences are expressed by the host cell.

### Embodiment 10

The method of embodiment 9, wherein the host cell is a mammalian cell, insect cell, bacterial cell, plant cell, viral cell, or fungal cell.

### Embodiment 11

The method of embodiment 1 or 2, wherein the malignancy exhibits predetermined immunoglobulin isotypes that are mixed, and wherein the vaccine comprises an IgM idiotype immunoglobulin.

### Embodiment 12

The method of embodiment 1 or 2, wherein the malignancy exhibits a predetermined immunoglobulin isotype that is only IgM, and wherein the vaccine comprises an IgM idiotype immunoglobulin.

### Embodiment 13

The method of embodiment 1 or 2, wherein the vaccine comprises an idiotype immunoglobulin that is produced by hybridoma rescue fusion hybridization.

### Embodiment 14

The method of embodiment 13, wherein the hybridoma is produced by fusion of a malignant B-cell obtained from the subject and a murine/human heterohybridoma myeloma cell.

### Embodiment 15

The method of embodiment 14, wherein the murine/human heterohybridoma myeloma cell is the K6H6/B5 cell line.

### Embodiment 16

The method of embodiment 1 or 2, wherein the vaccine comprises an idiotype immunoglobulin comprising at least an IgM constant region, and IgM variable region, wherein the idiotype immunoglobulin is produced recombinantly by introducing a genetic construct into a host cell, wherein the genetic construct comprises a nucleic acid sequence encoding the IgM constant region and a nucleic acid sequence the IgM variable region, and wherein the nucleic sequences are expressed by the host cell.

### Embodiment 17

The method of embodiment 16, wherein the host cell is a mammalian cell, insect cell, bacterial cell, plant cell, viral cell, or fungal cell.

### Embodiment 18

The method of any preceding embodiment, wherein the predetermined immunoglobulin isotype or isotypes exhibited by the malignancy represents an immunoglobulin that is present on the malignant cell (surface), within the malignant cell, secreted by the malignancy or is found in the subject's blood, or any combination of two or more of the foregoing.

### Embodiment 19

The method of any preceding embodiment, wherein the immunoglobulin isotype or isotypes exhibited by the malignancy is predetermined by obtaining a tumor, tissue or blood sample from the subject by biopsy (e.g., surgical biopsy or needle biopsy), needle aspiration, or apheresis.

### Embodiment 20

The method of any preceding embodiment, wherein the immunoglobulin isotype or isotypes exhibited by the malignancy is predetermined by obtaining a sample of lymph node tissue, extra-nodal tissue, spleen, bone marrow, or blood.

### Embodiment 21

The method of any preceding embodiment, wherein the immunoglobulin isotype or isotypes exhibited by the malignancy is predetermined by flow cytometry, immunofluoroescence, sequencing of heavy chain constant region, or immunoblot.

### Embodiment 22

The method of any one of embodiments 2-21, wherein said administering alleviates one or more symptoms associated with the B-cell malignancy.

### Embodiment 23

The method of any one of embodiments 2-22, wherein said administering prolongs remission duration in the subject.

### Embodiment 24

The method of any preceding embodiment, wherein the vaccine induces a humoral and/or a cellular immune response in the subject.

### Embodiment 25

The method of embodiment 24, wherein the immune response comprises both a cellular and humoral immune response.

### Embodiment 26

The method of any one of embodiments 2-25, further comprising assessing an immune response to the vaccine in the subject after said administering.

### Embodiment 27

The method of embodiment 26, wherein said assessing of the immune response to the vaccine comprises assessing the immune response against the B-cell idiotype.

### Embodiment 28

The method of embodiment 26 or 27, wherein the autologous idiotype immunoglobulin is linked to a carrier molecule (for example, keyhole limpet hemocyanin (KLH)), and wherein said assessing of the immune response to the vaccine comprises assessing the immune response against the B-cell idiotype and/or assessing the immune response against the carrier molecule.

### Embodiment 29

The method of embodiment 28, wherein said assessing of the immune response to the vaccine comprises both assessing the immune response against the B-cell idiotype and assessing the immune response against the carrier molecule.

### Embodiment 30

The method of any preceding embodiment, further comprising subsequently administering at least one booster dose of said vaccine to the subject.

### Embodiment 31

The method of any one of embodiments 2-30, further comprising comparing the immune response as assessed after said administering to an assessment of the immune response in the subject carried out before said administering.

### Embodiment 32

The method of any one of embodiments 2-31, wherein said assessing of the immune response to the vaccine is carried out multiple times at uniform or non-uniform time intervals, and further comprising comparing two or more assessments to determine whether the immune response to the vaccine has diminished.

### Embodiment 33

The method of embodiment 32, further comprising subsequently administering at least one additional booster dose of the vaccine to the subject if the immune response to the vaccine is determined to have diminished.

### Embodiment 34

The method of any one of embodiments 2-33, wherein the B-cell malignancy comprises a tumor, and said method further comprises assessing tumor response in the subject before said administering, after said administering, or before and after said administering.

### Embodiment 35

The method of any preceding embodiment, wherein the subject has undergone a different therapy for the malignancy prior to said administering.

### Embodiment 36

The method of embodiment 35, wherein the different therapy comprises chemotherapy and/or immunotherapy.

### Embodiment 37

The method of embodiment 35, wherein the different therapy comprises a monoclonal antibody.

### Embodiment 38

The method of embodiment 35, wherein the different therapy comprises a radioimmunotherapy.

### Embodiment 39

The method of embodiment 35, wherein the different therapy comprises a regimen of PACE (prednisone, doxorubicin, cyclophosphamide, and etoposide), CHOP (cyclophosphamide, doxorubicin, vincristine, and prednisone), CHOP-R (cyclophosphamide, doxorubicin, vincristine, prednisone, rituximab), B-R (bendamustine and rituximab), CVP (cyclophosphamide, vincristine, and prednisone), CVP-R (cyclophosphamide, vincristine, prednisone, and rituximab), F-R (fluradarabine and rituximab), FND-R (fludarabine, mitoxantrone, dexamethasone, and rituximab), FCM (fludarabine, cyclophosphamide, and mitoxantrone), FCM-R (fludarabine, cyclophosphamide, mitoxantrone, and rituximab), radioimmunotherapy (for example, ⁹⁰Y-ibritumomab tiuxetan or ¹³¹I-tositumomab), single agent rituximab, single agent alkylator (for example, chlorambucil or cyclophosphamide), lenalidomide, involved field radiation therapy, or stem cell transplant.

### Embodiment 40

The method of any one of embodiments 35-39, wherein the different therapy induces complete remission in the subject prior to the initial treatment with the autologous idiotype vaccine.

### Embodiment 41

The method of any preceding embodiment, wherein the subject is in complete remission at the time of said administering.

### Embodiment 42

The method of any preceding embodiment, wherein the B-cell malignancy is selected from the group consisting of non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, Burkitt lymphoma/leukemia.

### Embodiment 43

The method of any preceding embodiment, wherein the B-cell malignancy is a mature B-cell lymphoma.

### Embodiment 44

The method of any preceding embodiment, wherein the B-cell malignancy is a mature B-cell lymphoma selected from the group consisting of B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, Burkitt lymphoma/Burkitt cell leukemia.

### Embodiment 45

The method of any preceding embodiment, wherein the subject has reduced T-regulatory cell activity and/or reduced numbers of T-regulatory cells at the time of administration of the vaccine.

### Embodiment 46

The method of embodiment 45, wherein the reduced T-regulatory cell activity and/or reduced numbers of T-regulatory cells is achieved by administration of a T-regulatory cell inhibitor to the subject prior to administration of the vaccine.

### Embodiment 47

The method of embodiment 46, wherein the T-regulatory cell inhibitor is selected from among lenalidomide, pomalidomide, an oxazaphosphorine (for example, cyclophosphamide), anti-CD25 monoclonal antibody, IL-2Ra monoclonal antibody, and anti-GITR monoclonal antibody.

### Embodiment 48

The method of any preceding embodiment, wherein the subject is human.

### Embodiment 49

A method for selecting a treatment for a subject having a B-cell malignancy, comprising screening the patient for a heavy-chain isotype, wherein if the isotype has detectable M isotype (for example, IgM, IgM+IgG, or IgM+IgH (wherein IgH refers generically to any heavy chain)) production of an autologous idiotype IgM vaccine is authorized and treatment of the subject with the autologous idiotype IgM vaccine can (and preferably does) proceed; and

wherein if the subject has only a non-IgM B-cell malignancy, (a) production of a recombinant idiotype vaccine for the subject (Idiotype+IgM) is authorized and treatment of the subject with the recombinant vaccine can proceed; or optionally (b) the subject is excluded from treatment with an idiotype vaccine and an alternative treatment with an alternative (non-idiotype vaccine) therapy is authorized (for example, rituximab+chemotherapy: R-CHOP, R-CVP or PACE, or chlorambucil-containing chemotherapy, or autologous stem cell transplant) and may (and preferably does) proceed.

### Embodiment 50

The method of embodiment 49, further comprising administering the autologous idiotype IgM vaccine to the subject.

### Embodiment 51

The method of embodiment 49, further comprising administering the recombinant idiotype vaccine to the subject.

### Embodiment 52

The method of embodiment 49, further comprising administering the alternative therapy to the subject.

### Embodiment 53

The method of any preceding embodiment, wherein the autologous idiotype immunoglobulin is linked to a carrier molecule.

### Embodiment 54

The method of embodiment 53, wherein the carrier molecule comprises keyhole limpet hemocyanin (KLH).

### Embodiment 55

The method of embodiment 1 or 2, wherein the autologous idiotype immunoglobulin is not linked to a carrier molecule.

### Embodiment 56

The method of embodiment 2, further comprising administering an adjuvant to the subject before, simultaneously with, or after administering the autologous idiotype vaccine.

### Embodiment 57

The method of embodiment 56, wherein the adjuvant comprises granulocyte-monocyte colony-stimulating factor (GM-CSF).

### Embodiment 58

The method of embodiment 2, wherein the method does not include administration of an adjuvant.

### Embodiment 59

A method for preparing an autologous anti-idiotype vaccine for treatment of a B-cell malignancy in a subject, said method comprising preparing an autologous anti-idiotype vaccine for the subject, wherein the vaccine comprises an autologous anti-idiotype immunoglobulin (Ab2 immunoglobulin) comprising at least an IgM constant region, wherein the Ab2 immunoglobulin is directed against an idiotype of an immunoglobulin (Ab1 immunoglobulin), and wherein the Ab1 immunoglobulin is specific for the idiotype of the B-cell malignancy.

### Embodiment 60

A method for treating a B-cell malignancy in a subject, comprising administering an autologous anti-idiotypic vaccine to the subject, wherein the vaccine comprises an autologous anti-idiotype immunoglobulin (Ab2 immunoglobulin) comprising at least an IgM constant region, wherein the Ab2 immunoglobulin is directed against an idiotype of an immunoglobulin (Ab1 immunoglobulin), and wherein the Ab1 immunoglobulin is specific for the idiotype of the B-cell malignancy.

### Embodiment 61

A method for selecting a treatment for a B-cell malignancy, comprising determining the T-regulatory (T-reg) cell level (T-reg cell number and/or T-reg activity) in the subject; wherein if the T-reg cell level is consistent with a normal T-reg cell level, an effective amount of a T-reg cell inhibitor is administered to the subject prior to administration of a vaccine of any preceding embodiment.

### Embodiment 62

The method of embodiment 61, wherein said determining comprising obtaining at least one biological sample (for example, blood) from the subject and deteiinining the T-reg cell level in the sample.

### Embodiment 63

The method of embodiment 61, wherein the T-reg cell inhibitor is administered to the subject until the T-reg cell level in the subject is below that of an immunosuppressive T-reg cell level.

### Embodiment 64

The method of embodiment 61, wherein the T-reg cell level is determined two or more times and the T-reg cell inhibitor is administered to the subject until the T-reg cell level in the subject is below that of a immunosuppressive T-reg cell level, prior to administration of the vaccine.

### Embodiment 65

The method of embodiment 61, wherein said determining of the T-reg cell level comprises carrying out flow cytometry on a biological sample obtained from the subject.

### Embodiment 66

The method of embodiment 65, wherein said determining of the T-reg cell level comprises carrying out flow cytometry on a biological sample obtained from the subject to quantitate the number of CD4+ CD25HIFoxp3+ cells in the biological sample. Embodiment 67

A kit for treatment of a B-cell malignancy, comprising at least one autologous idiotype vaccine and printed instructions for using the vaccine for treatment of the B-cell malignancy.

### Embodiment 68

The kit of embodiment 67, further comprising an immune adjuvant.

### Embodiment 69

The kit of embodiment 67, further comprising one or more reagents for assessing immune response in a subject.

### Embodiment 70

The kit of embodiment 67, wherein the idiotype vaccine comprises an autologous idiotype immunoglobulin linked to a carrier molecule.

### Embodiment 71

A kit for assessing a humoral response to a vaccine of any preceding embodiment, comprising an assay for detection of anti-idiotype immunoglobulins in a sample through their capacity to bind to the vaccine idiotype, and printed instructions for using the assay to detect the humoral response.

### Embodiment 72

The kit of embodiment 71, wherein said assay is an enzyme-linked immunsorbent assay (ELISA).

### Embodiment 73

The kit of embodiment 72, wherein said ELISA is a colorimetric, chemiluminescent, fluorescent, or radioactive assay.

### Embodiment 74

A kit for assessing a cellular response to a vaccine of any preceding embodiment, comprising an assay for detection of one or more activation markers, cytokines, growth factors, or cell subsets indicative of a cellular response, or a combination of two or more of the foregoing.

### Embodiment 75

The kit of embodiment 74, wherein said assay is an enzyme-linked immunsorbent assay (ELISA).

### Embodiment 76

The kit of embodiment 75, wherein said ELISA is a colorimetric, chemiluminscent, fluorescent, or radioactive assay.

### Embodiment 77

A kit for detecting the T-regulatory (T-reg) cell response before, during, and after administration of a T-reg inhibitor prior to administration of a vaccine of any preceding embodiment, wherein said kit comprises one or more reagents for assessing T-reg cell response in a subject; and printed instructions for making the assessment.

### Embodiment 78

The kit of embodiment 77, further comprising a T-reg cell inhibitor.

### Embodiment 79

An autologous idiotype vaccine of any preceding embodiment.

### Embodiment 80

The vaccine of embodiment 79, wherein the vaccine comprises a chimeric idiotype immunoglobulin comprising at least an IgM constant region, and a variable region derived from a non-IgM immunoglobulin expressed by a B-cell malignancy.

### Embodiment 81

The vaccine of embodiment 79, wherein the vaccine comprises a chimeric idiotype immunoglobulin comprising at least an IgM constant region, and a variable region derived from an IgM immunoglobulin expressed by a B-cell malignancy.

### Embodiment 82

The vaccine of embodiment 79, wherein the vaccine comprises a chimeric idiotype immunoglobulin that is produced recombinantly by introducing a genetic construct into a host cell, wherein the genetic construct comprises a nucleic acid sequence encoding the IgM constant region and a nucleic acid sequence coding for the variable region of the immunoglobulin expressed by the malignant cell wherein the isotype of the immunoglobulin is not IgM, and wherein the nucleic sequences are expressed by the host cell.

### Embodiment 83

A composition comprising an autologous idiotype vaccine of embodiment 79, further comprising one or more anti-cancer compounds.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Vaccination with Patient-Specific Hybridoma-Derived ID Protein Vaccine Prolongs Disease-Free Survival in Follicular Lymphoma Patients

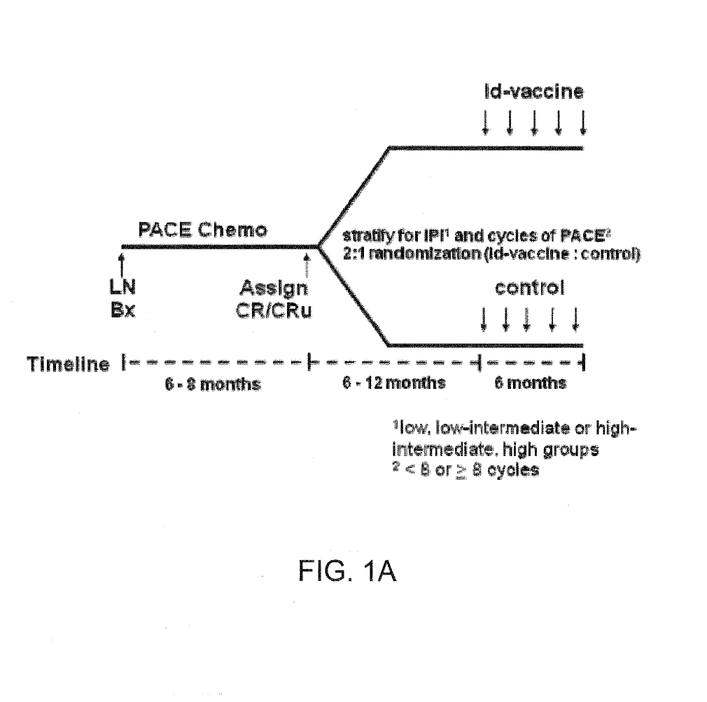

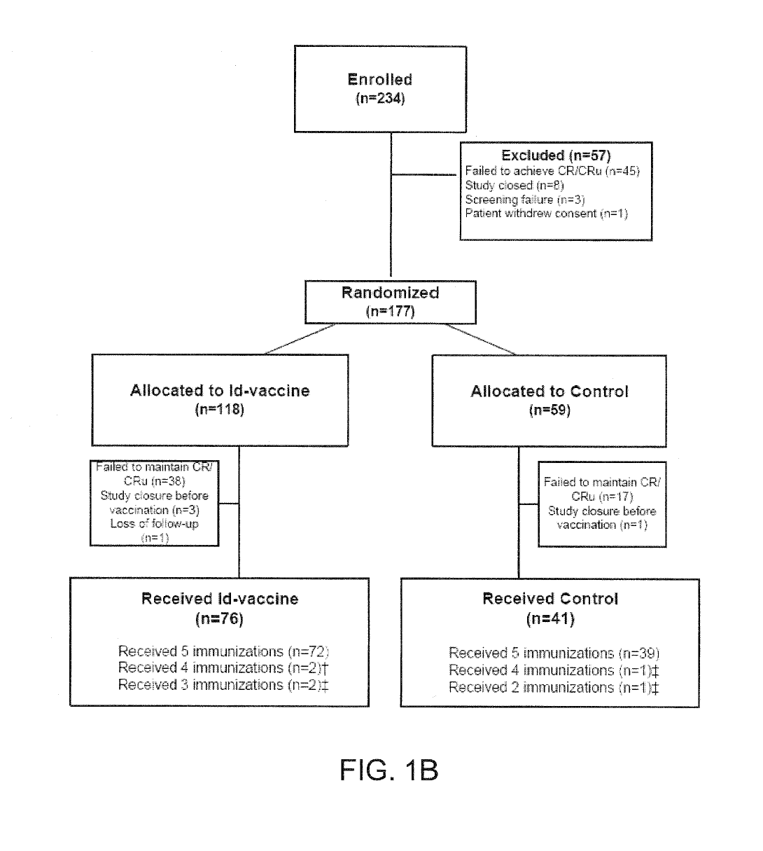

Starting in January 2000, a total of 234 patients were enrolled in the study (FIG. 1B and Table 4). Due to protracted enrollment (Table 5), the trial was terminated prior to full accrual and the data were locked on Jun. 30, 2008, following DMC recommendation. At study termination, 219 patients completed PACE chemotherapy and 6 completed cyclophosphamide, doxorubicin, vincristine, prednisone, and rituximab (CHOP-R) chemotherapy. Of the patients who received PACE, 177 (81%) achieved CR/CRu, and were stratified and randomized to receive either Id-vaccine (n=118) or control (n=59) (Table 4). Fifty-seven (24%) patients were excluded from randomization because of failing to achieve CR/CRu (n=45), study closure (n=8), screening failure (n=3), or withdrawing consent (n=1). Patients who received CHOP-R were among the 57 patients excluded either due to study closure (n=3) or for failing to achieve CR/CRu (n=3). Prior to vaccination, 55 (31%) randomized patients relapsed (38 in the Id-vaccine aim, 17 in the control arm); and 5 randomized patients were excluded due to study closure (3 in the Id-vaccine arm, 1 in the control arm) or loss of follow-up (1 in the Id-vaccine arm). Of the 117 patients who received at least one blinded vaccination, 76 received Id-vaccine and 41 received control. As expected from the vaccine release algorithm, the median time between randomization and initiation of vaccinations was not significantly different between the Id-vaccine (8.74 months) and control (8.31 months) arms (P=0.279). Idiotype protein was successfully produced in 72 of 76 patients (95%) assigned to receive the Id-vaccine. Five patients assigned to the experimental aim received KLH+GM-CSF due to failure to make Id protein but were analyzed as randomized. Six patients did not complete the five intended vaccinations either due to withdrawal (n=2) or relapsed disease (n=4) but were analyzed as randomized. All baseline characteristics were well balanced between the groups that received blinded vaccinations (n=117) (Table 1) as well as between the two groups of the 60 randomized patients that did not receive vaccinations (Table 2).

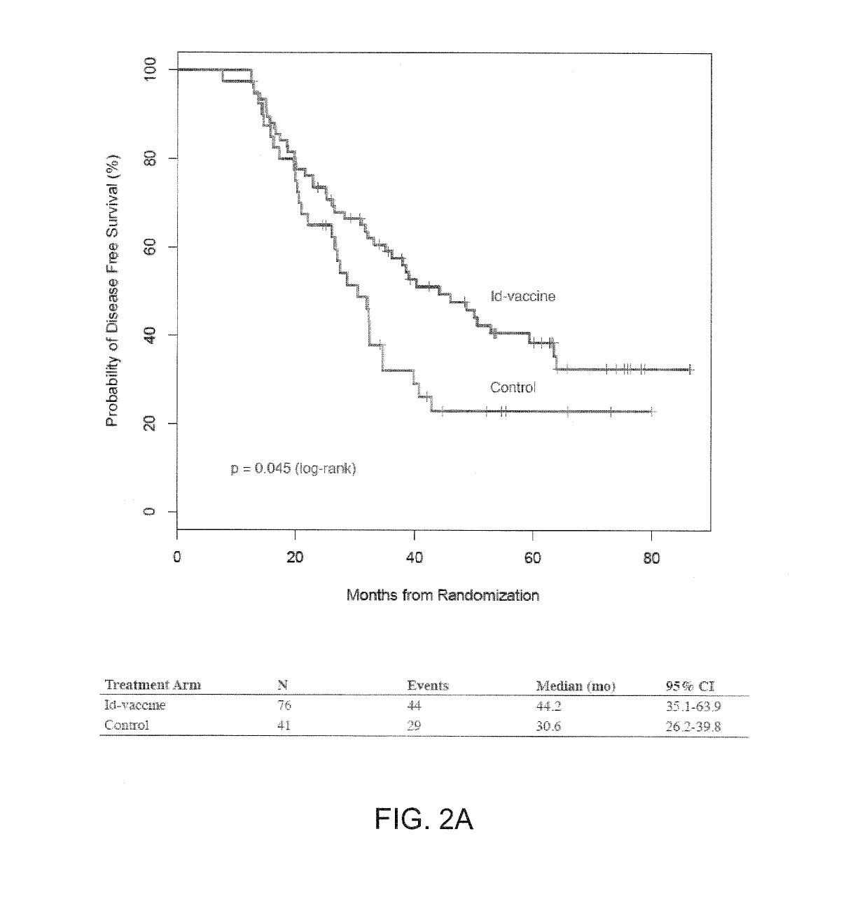

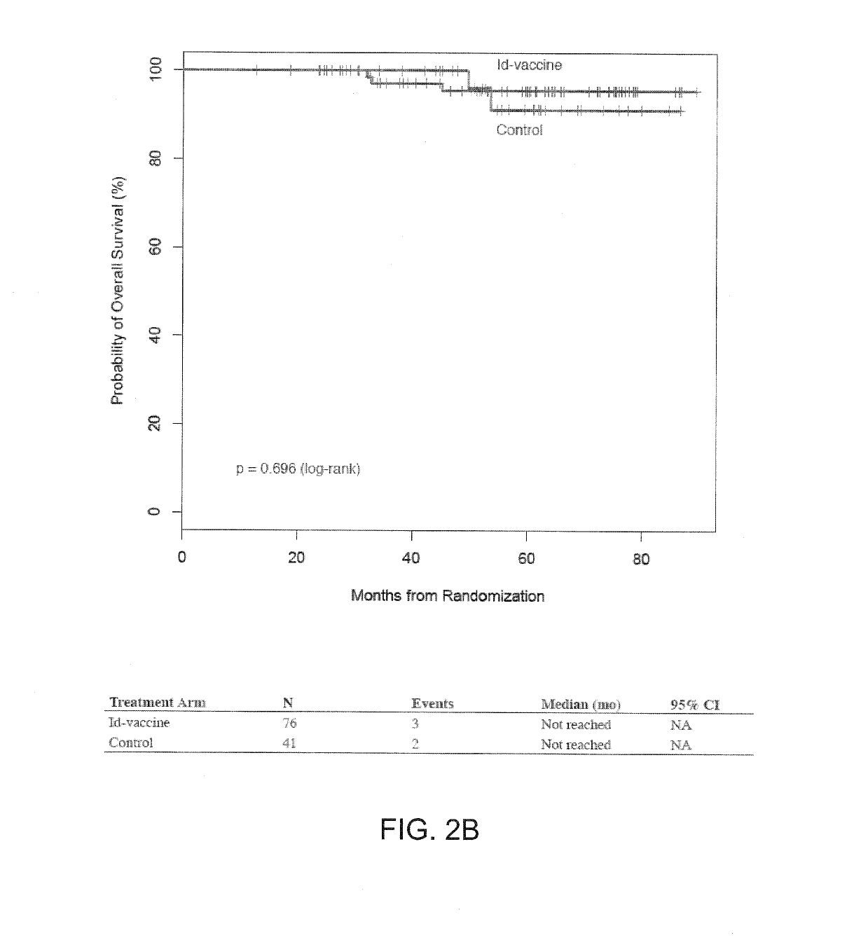

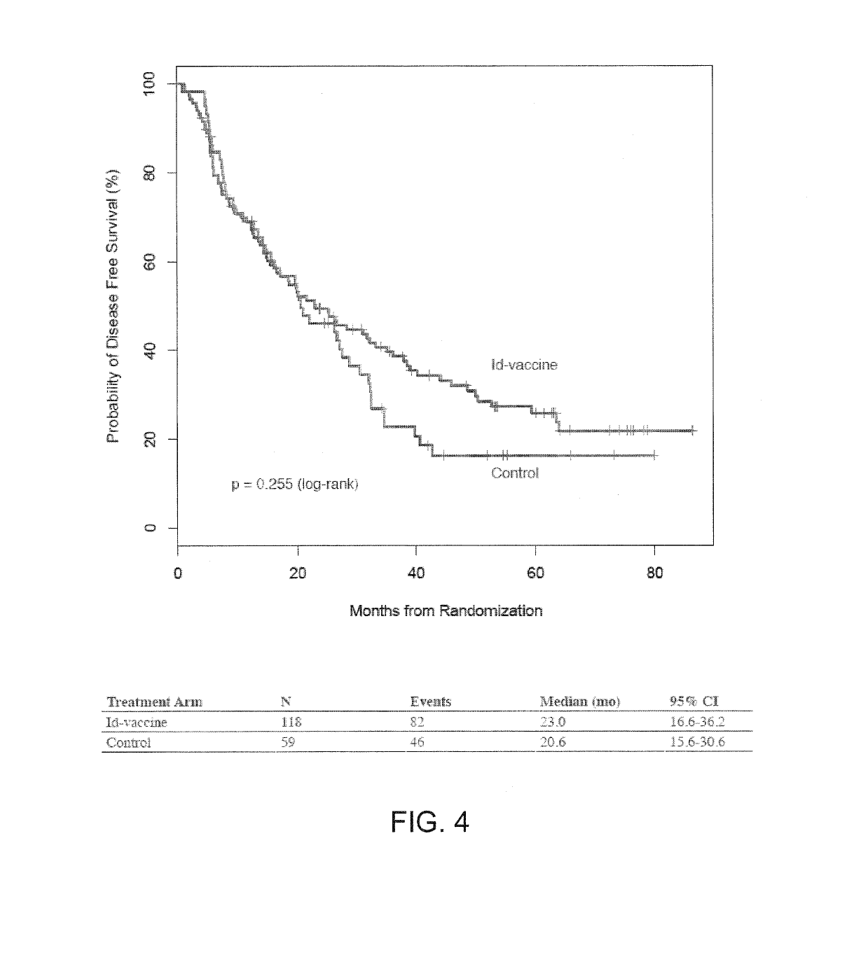

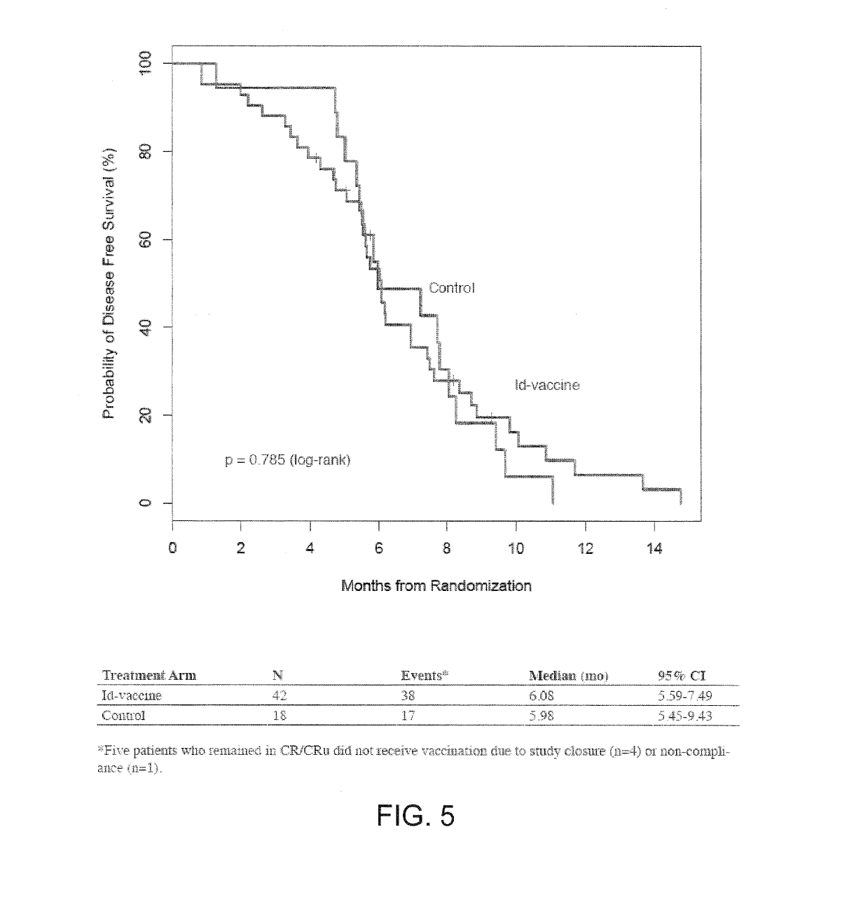

For the 117 patients who received at least one blinded vaccination, median DFS was significantly prolonged in the Id-vaccine arm compared to the control arm (FIG. 2A). At a median follow-up of 56.6 months (range 12.6-89.3 months), median DFS after randomization to the Id-vaccine arm was 44.2 months versus 30.6 months for the control arm (P=0.045). Using Cox proportional hazard model, a statistically-significant hazard ratio (HR) of 0.62 was achieved (P=0.047; 95% confidence interval [CI]: 0.39-0.99). Median overall survival (OS) was not reached for either group; the number of deaths were too few to enable any conclusions about overall survival (FIG. 2B). For all 177 randomized patients, median DFS from randomization between the Id-vaccine and control arms was 23.0 vs. 20.6 months, respectively (P=0.256; HR=0.81; 95% CI: 0.56-1.16) (FIG. 4). There was no statistically-significant difference in median DFS between arms for the 60 randomized patients who did not receive vaccinations (6.08 months for Id-vaccine arm vs. 5.98 months for control arm; P=0.78; HR=0.92; 95% CI: 0.51-1.65) (FIG. 5) suggesting that the arms were well balanced for baseline characteristics (Table 2). Analysis of the group of 117 patients who received at least one blinded vaccination showed statistically-significant improvement in DFS in the Id-vaccine arm compared to the control arm (FIG. 2A).

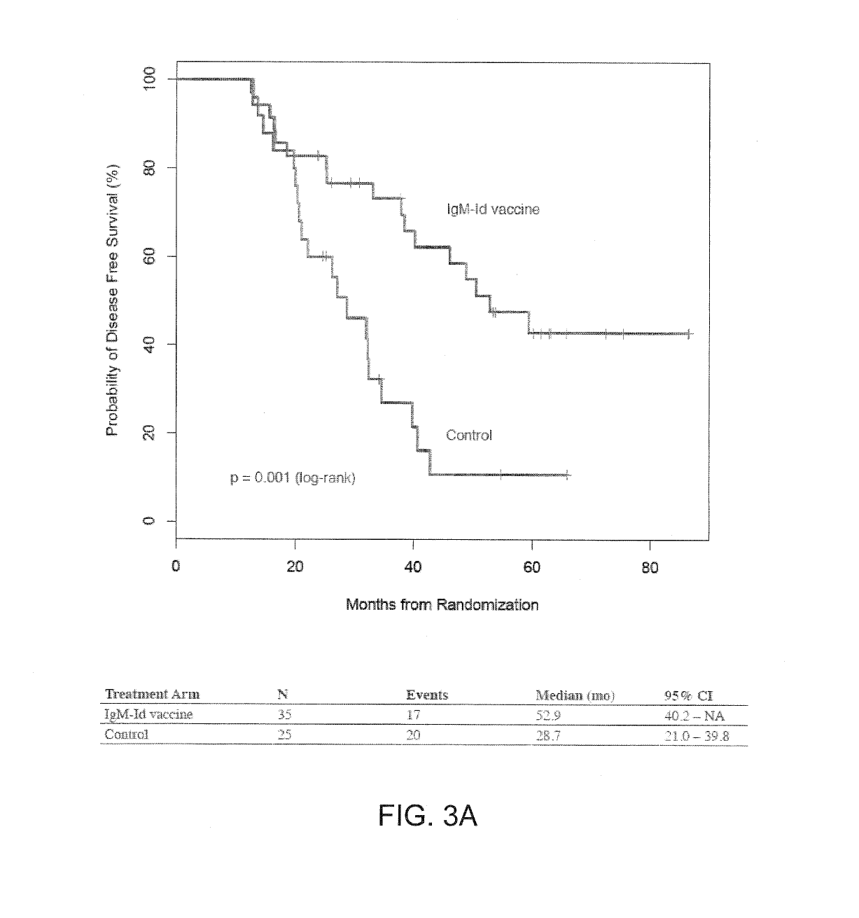

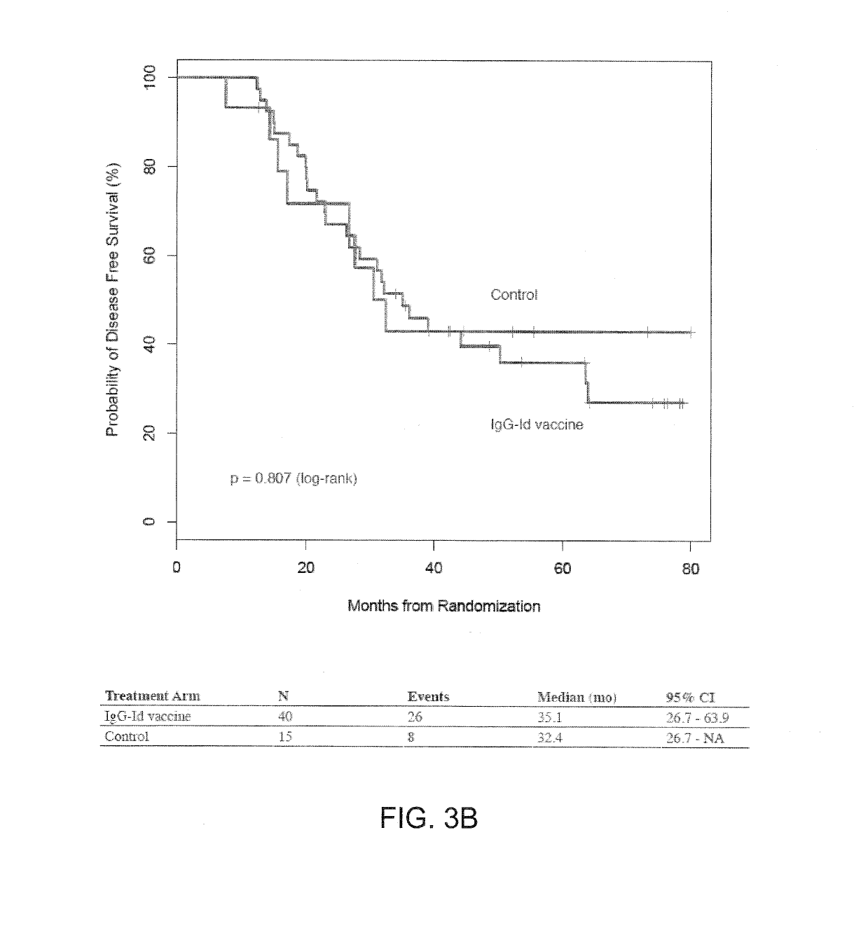

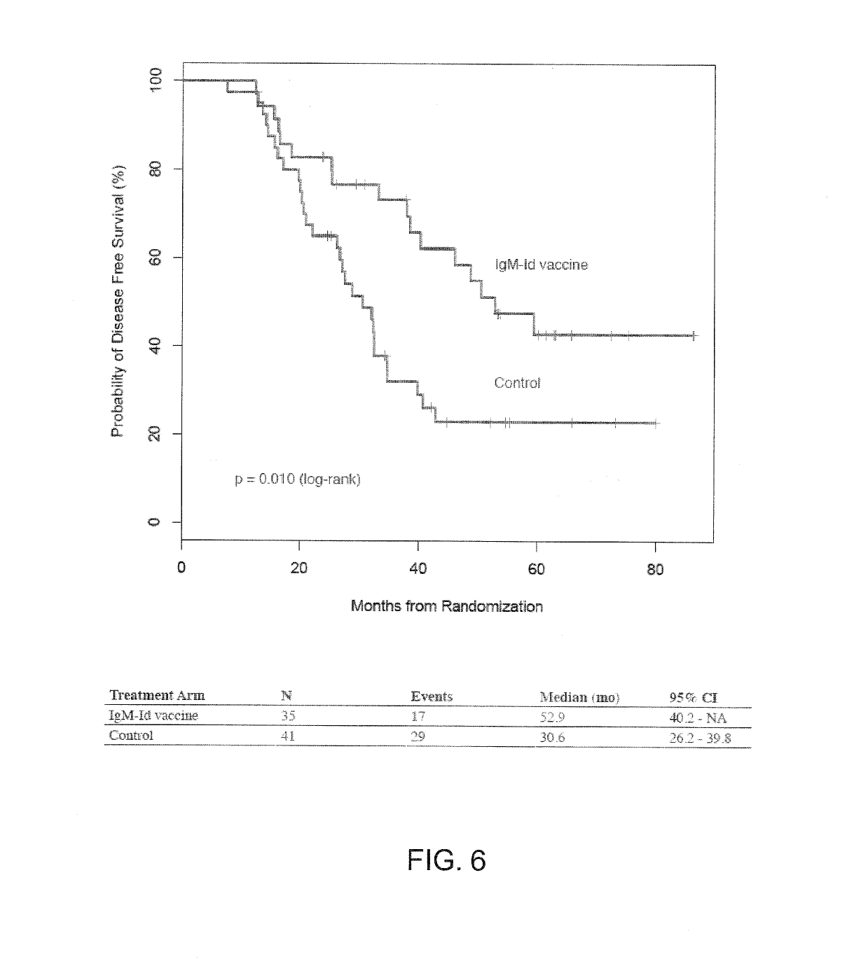

DFS of vaccinated patients was also analyzed by tumor Ig heavy- and light-chain isotypes. For IgM and IgG heavy-chain isotype groups, there were no statistically significant differences in baseline patient characteristics between experimental and control arms (n=35 vs. 25 for IgM isotype and n=40 vs. 15 for IgG isotype for Id-vaccine and control arms, respectively). Two patients had mixed IgM/IgG biopsy isotypes and were excluded from this analysis (Tables 4 and 6). Among patients receiving an IgM-Id vaccine, median time to relapse after randomization was 52.9 months, versus 28.7 months in the IgM tumor isotype control-treated patients (p=0.001; HR=0.34 [p=0.002]; 95% CI:0.17-0.68) (FIG. 3A) and 30.6 months in all controls (p=0.010: FIG. 6). Among patients receiving an IgG-Id vaccine, median time to relapse after randomization was 35.1 months, versus 32.4 months in the IgG tumor isotype control-treated patients (p=0.807; HR=1.1 [p=0.807]; 95% CI-0.50-2.44) (FIG. 3B). Cox proportional hazard modeling supports an interaction between treatment and Ig isotype (p=0.039). When patients were grouped by light chain type, there was no difference in DFS (data not shown).

Both Id-vaccine and control were safe and well-tolerated. There were no statistically significant differences in frequency or types of AE observed between groups. Grade 1-2 AE were common in both groups (Table 7). However, grade 3-4 AE were rare; there were no Id-vaccine-related deaths (Table 3). The most common AE were injection site reactions (>80% of patients on each arm) with erythema and induration lasting for a few days after each vaccination.

This controlled clinical trial demonstrates that vaccination with patient-specific hybridoma-derived Id protein vaccine prolongs DFS, compared to controls, in FL patients vaccinated during a CR/CRu lasting at least six months after PACE chemotherapy. The principal focus of the efficacy analysis was on the group of patients receiving at least one blinded vaccination. For this patient group, the results showed a statistically significant improvement in DFS following Id vaccination, compared with the control arm (FIG. 2A). In general, the ideal time for randomization is at the time of initiating experimental therapy. However, the decision was made the decision to randomize well in advance, immediately after completion of chemotherapy, so that resources would not be expended manufacturing patient-specific vaccines for the control group. Nevertheless, the conclusions should have the same validity as if randomization had occurred at initial vaccination, with the principal potential concern that patients in one arm may be more likely to drop out of the study before vaccination. Indeed, DFS analysis of the 60 patients who were randomized but not vaccinated showed no suggestion of treatment effect (FIG. 5), demonstrating that the arms were well balanced for baseline characteristics (Table 2). Furthermore, the concealed randomization, the double-blinded nature of the study, the use of a vaccine release algorithm to achieve comparable time from randomization to vaccination, the similar rate of injection site reactions in both groups (Table 3), and the analysis of data by an independent statistician guarded against the introduction of unintentional bias in the efficacy analysis of the 117 vaccinated patients. The improvement in DFS with the Id-vaccine (FIG. 2A) despite the use of KLH±GM-CSF, a potentially active form of immunotherapy,^{19,20} in the control arm also suggests that the clinical benefit induced by the Id-vaccine may have been even greater had the control group received a placebo. The treatment comparison for all 177 randomized 15 patients was not statistically significant (FIG. 4) because inclusion of the 60 non-vaccinated patients obscured the treatment effect shown in FIG. 2A.

Although termination of the trial before completion of the planned accrual resulted in a smaller sample size than originally intended and decreased the power to detect a difference in DFS between treatment arms, the study, nevertheless, showed a statistically significant improvement in DFS for Id vaccinated patients (FIG. 2A). As previously suggested, randomized trials may overcome limitations of small sample size and yield valid conclusions if baseline characteristics are well balanced, allocation is concealed, and they are double-blinded.^{21,22} These features, built into this trial, together with the fact that the HR for DFS is 0.62 (FIG. 2A), support the conclusion that the treatment effect observed by this vaccine was not exaggerated.

To determine whether isotype of the surface immunoglobulin used as an idiotype vaccine influences clinical response, DFS of vaccinated patients was analyzed according to their tumor Ig isotype. The inventors observed that patients immunized with IgM-Id vaccines had significantly longer DFS than control patients with IgM isotype tumors, while DFS for those receiving IgG-Id vaccines did not differ from isotype-matched controls (FIG. 3). Although this trial was not powered to address such subset analysis and this analysis was not pre-specified in the protocol, the observed treatment effects differ dramatically by isotype. While the epitopes after Id vaccination have been shown to be derived from the unique variable region of the tumor's immunoglobulin,^{25,26} the isotype of the constant region may influence the immunogenicity of variable region epitopes.^{23,24} Preclinical studies have shown that Ids that have switched to IgG became tolerogenic, while Ids of their IgM progenitors were highly immunogenic.^{23,24} The improvement in DFS observed for patients receiving Id-vaccine in this trial stands in contrast to the results of the Genitope²⁷ and Favrille²⁸ phase III trials, which failed to show clinical benefit with recombinant tumor-derived Id vaccines in FL. The significant differences in trial design and vaccine formulation are likely responsible for the different clinical outcomes observed in these three phase 3 trials (Table 8). The present study used the phase II NCI treatment protocol and the hybridoma Id protein manufacturing method.^{12,18} With regard to trial designs, the Favrille and Genitope trials differed significantly from this trial by extending eligibility to patients with partial response and stable disease in addition to CR/Cru after chemotherapy, using less aggressive induction chemotherapy prior to vaccination, and not stratifying by clinical prognostic factors for treatment allocation. It is conceivable that the benefit of Id vaccination is discernable only in patients with minimal residual disease (CR/CRu) after chemotherapy. The hybridoma technique¹⁸ used in this trial yields Id proteins that more closely resembled the native Ig on the tumor cell surface, compared with the recombinant DNA-derived Id proteins used in the Genitope and Favrille studies.¹⁰ Production of recombinant protein may have altered post-translational modifications such as glycosylation, which can result in profound changes in final protein tertiary structure.²⁹ In addition, the hybridoma technique yields Id proteins with IgM or IgG Fc regions identical to the tumor Ig isotype as opposed to a universal IgG Fc used to produce Id vaccines for all patients in the Genitope and Favrille trials. It is possible that the use of a universal IgG Fc may have altered the immunogenicity of the Id vaccine (FIG. 3). This trial was initiated in the pre-rituximab era and used standard combination chemotherapy as the induction regimen. In current practice, chemotherapy is administered with rituximab, an anti-CD20 monoclonal antibody shown to improve overall response rate, progression-free survival, and overall survival in FL patients.^{2,3,30,31} However, rituximab-containing immunochemotherapies do not appear to be curative and complementary treatment strategies are needed.^{30,31} Although rituximab induces prolonged B-cell deletion and impairs induction of humoral responses following Id vaccination, generation of tumor-specific cellular immunity is not affected.³² Phase I and II clinical trials suggest that tumor-specific humoral and cellular immune responses after Id vaccination may each independently induce tumor regression and have been associated with improvement in clinical outcome in FL.^{10-12,33,34} While the relative importance of humoral versus cellular immunity in the efficacy of Id vaccination is unclear, cellular immunity induced by Id vaccination could, conceptually, complement rituximab-containing immunochemotherapies.²⁵

This trial proves the principle that therapeutic vaccination can result in meaningful clinical benefit for FL patients by prolonging DES. Furthermore, the results of this trial suggest that the isotype of the constant region may influence the immunogenicity of Id vaccines. This finding could have profound implications on Id vaccine production strategies and clinical development for FL and other B-cell malignancies.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. A clinical evaluation of the International Lymphoma Study Group classification of non-Hodgkin's lymphoma. The Non-Hodgkin's Lymphoma Classification Project. Blood 89:3909-18 (1997).
2. Hiddemann W, Kneba M, Dreyling M, et al: Frontline therapy with rituximab added to the combination of cyclophosphamide, doxorubicin, vincristine, and prednisone (CHOP) significantly improves the outcome for patients with advanced-stage follicular lymphoma compared with therapy with CHOP alone: results of a prospective randomized study of the German Low-Grade Lymphoma Study Group. Blood 106:3725-32 (2005).
3. Marcus R, Imrie K, Solal-Celigny P, et al: Phase III study of R-CVP compared with cyclophosphamide, vincristine, and prednisone alone in patients with previously untreated advanced follicular lymphoma. J Clin Oncol 26:4579-86 (2008).
4. Stevenson, G. T. & Stevenson, F. K. Antibody to a molecularly-defined antigen confined to a tumour cell surface. Nature 254, 714-716 (1975).
5. Sirisinha, S. & Eisen, H. N. Autoimmune-like antibodies to the ligand-binding sites of myeloma proteins. Proc Natl Acad Sci USA 68, 3130-3135 (1971).
6. Lynch, R. G., Graff, R. J., Sirisinha, S., Simms, E. S. & Eisen, H. N. Myeloma proteins as tumor-specific transplantation antigens. Proc Natl Acad Sci USA 69, 1540-1544 (1972).
7. Kaminski, M. S., Kitamura, K., Maloney, D. G. & Levy, R. Idiotype vaccination against murine B cell lymphoma. Inhibition of tumor immunity by free idiotype protein. J Immunol 138, 1289-1296 (1987).
8. Kwak, L. W., Young, H. A., Pennington, R. W. & Weeks, S. D. Vaccination with syngeneic, lymphoma-derived immunoglobulin idiotype combined with granulocyte/macrophage colony-stimulating factor primes mice for a protective T-cell response. Proc Natl Acad Sci USA 93, 10972-10977 (1996).
9. Kwak, L. W., et al. Induction of immune responses in patients with B-cell lylnphoma against the surface-immunoglobulin idiotype expressed by their tumors. N Engl J Med 327, 1209-1215 (1992).
10. Park, H. J. & Neelapu, S. S. Developing idiotype vaccines for lymphoma: from preclinical studies to phase III clinical trials. Br J Haematol 142, 179-191 (2008).
11. Houot, R. & Levy, R. Vaccines for lymphomas: idiotype vaccines and beyond. Blood Rev 23, 137-142 (2009).
12. Bendandi, M., et al. Complete molecular remissions induced by patient-specific vaccination plus granulocyte-monocyte colony-stimulating factor against lymphoma. Nat Med 5, 1171-1177 (1999).
13. Cheson B D, Horning S J, Coiffier B, et al: Report of an international workshop to standardize response criteria for non-Hodgkin's lymphomas. NCI Sponsored International Working Group. J Clin Oncol 17:1244 (1999).
14. Longo D L, DeVita V T, Jr., Duffey P L, et al: Superiority of ProMACE-CytaBOM over ProMACE-MOPP in the treatment of advanced diffuse aggressive lymphoma: results of a prospective randomized trial. J Clin Oncol 9:25-38 (1991).
15. Longo D L, Duffey P L, Gribben J G, et al: Combination chemotherapy followed by an immunotoxin (anti-B4-blocked ricin) in patients with indolent lymphoma: results of a phase II study. Cancer J 6:146-50 (2000).
16. A predictive model for aggressive non-Hodgkin's lymphoma. The International. Non-Hodgkin's Lymphoma Prognostic Factors Project. N Engl J Med 329:987-94 (1993).
17. Coiffier B, Lepage E, Briere J, et al: CHOP chemotherapy plus rituximab compared with CHOP alone in elderly patients with diffuse large-B-cell lymphoma. N Engl J Med 346:235-42 (2002).
18. Carroll W L, Thielemans K, Dilley J, et al: Mouse×human heterohybridomas as fusionpartners with human B cell tumors. J Immunol Methods 89:61-72 (1986).
19. Jones S E, Schottstaedt M W, Duncan L A, et al: Randomized double-blind prospective trial to evaluate the effects of sargramostim versus placebo in a moderate-dose fluorouracil, doxorubicin, and cyclophosphamide adjuvant chemotherapy program for stage II and III breast cancer. J Clin Oncol 14:2976-83 (1996).
20. Spitler L E, Grossbard M L, Ernstoff M S, et al: Adjuvant therapy of stage III and IV malignant melanoma using granulocyte-macrophage colony-stimulating factor. J Clin Oncol 18:1614-21 (2000).
21. Schulz K F, Chalmers I, Hayes R J, et al: Empirical evidence of bias. Dimensions of methodological quality associated with estimates of treatment effects in controlled trials. JAMA 273:408-12 (1995).
22. Kjaergard L L, Villumsen J, Gluud C: Reported methodologic quality and discrepancies between large and small randomized trials in meta-analyses. Ann Intern Med 135:982-9 (2001).
23. Reitan S K, Hannestad K: A syngeneic idiotype is immunogenic when borne by IgM but tolerogenic when joined to IgG. Eur J Immunol 25:1601-8 (1995).
24. Reitan S K, Hannestad K: Immunoglobulin heavy chain constant regions regulate immunity and tolerance to idiotypes of antibody variable regions. Proc Natl Acad Sci USA 99:7588-93 (2002).
25. Baskar S, Kobrin C B, Kwak L W: Autologous lymphoma vaccines induce human T cell responses against multiple, unique epitopes. J Clin Invest 113:1498-510 (2004).
26. Bertinetti C, Zirlik K, Heining-Mikesch K, et al: Phase I trial of a novel intradermal idiotype vaccine in patients with advanced B-cell lymphoma: specific immune responses despite profound immunosuppression. Cancer Res 66:4496-502 (2006).
27. Levy R R M, Leonard J, Vose J, Denney D.: Results of a phase 3 trial evaluating safety and efficacy of specific immunotherapy, recombinant idiotype (Id) conjugated to KLH (Id-KLH) with GM-CSF, compared with non-specific immunotherapy, KLH with GM-CSF, in patients with follicular non Hodgkin's lymphoma (FNHL). Annals of Oncology 19:Suppl 4:iv101-102 (2008).
28. Freedman A, Neelapu S S, Nichols C, et al: Placebo-controlled phase III trial of patientspecific immunotherapy with mitumprotimut-T and granulocyte-macrophage colonystimulating factor after rituximab in patients with follicular lymphoma. J Clin Oncol 27:3036-43 (2009).
29. Redfern C H, Guthrie T H, Bessudo A, et al: Phase II trial of idiotype vaccination in previously treated patients with indolent non-Hodgkin's lymphoma resulting in durable clinical responses. J Clin Oncol 24:3107-12 (2006).
30. Fisher R1, LeBlanc M, Press O W, et al: New treatment options have changed the survival of patients with follicular lymphoma. J Clin Oncol 23:8447-52 (2005).
31. Liu Q, Fayad L, Cabanillas F, et al: Improvement of overall and failure-free survival in stage 1V follicular lymphoma: 25 years of treatment experience at The University of Texas M.D. Anderson Cancer Center. J Clin Oncol 24:1582-9 (2006).
32. Neelapu S S, Kwak L W, Kobrin C B, et al: Vaccine-induced tumor-specific immunity despite severe B-cell depletion in mantle cell lymphoma. Nat Med 11:986-91 (2005).
33. Weng W K, Czerwinski D, Timmerman J, et al: Clinical outcome of lymphoma patients after idiotype vaccination is correlated with humoral immune response and immunoglobulin G Fc receptor genotype. J Clin Oncol 22:4717-24 (2004).
34. Nelson E L, Li X, Hsu F J, et al: Tumor-specific, cytotoxic T-lymphocyte response after idiotype vaccination for B-cell, non-Hodgkin's lymphoma. Blood 88:580-9 (1996).

TABLE 1
Characteristics of randomized patients who received
vaccination (N = 117)

| | Id-vaccine | Control | |
|---|---|---|---|
| | (N = 76) | (N = 41) | |
| Characteristic | No. (%) | No. (%) | P value^{#} |
| Age at enrollment -years | 49.7 ± 9.7* | 51.7 ± 9.1* | 0.146 |
| Male sex | 39 (51.3) | 28 (68.3) | 0.083 |
| White race group | 67 (88.2) | 38 (92.7) | 0.537 |
| ECOG Performance Status | | | 0.222 |
| 0 | 64 (84.2) | 30 (73.2) | |
| 1 | 12 (15.8) | 11 (26.8) | |
| Histology | | | 0.845 |
| FL, grade 1 | 34 (44.7) | 17 (41.5) | |
| FL, grade 2 | 42 (55.3) | 24 (58.5) | |
| IgM isotype | 35 (46.1) | 25 (61.0) | |
| IgG isotype | 40 (52.6) | 15 (36.6) | |
| IgM/IgG isotype | 1 (1.3) | 1 (2.4) | |
| Stage | | | 0.263 |
| II | 2 (2.6) | 1 (2.4)^{a} | |
| III | 29 (38.2) | 10 (24.4)^{b} | |
| IV | 45 (59.2) | 30 (73.2)^{c} | |
| International Prognostic Index | | | 1.000 |
| Low or low intermediate (0-2) | 69 (90.8) | 37 (90.2) | |
| High intermediate or high (3-5) | 7 (9.2) | 4 (9.8) | |
| ≦8 induction chemotherapy cycles | 38 (50.0) | 22 (53.7) | 0.846 |

*Plus-minus values are means ± SD.
^{a}P = 1.000 for the comparison for stage II representation between the two arms.
^{b}P = 0.154 for the comparison for stage III representation between the two arms.
^{c}P = 0.160 for the comparison for stage IV representation between the two arms.
^{#}Comparisons between age groups were performed with non-parametric t-tests using the normal approximation (two-sided Wilcoxon test). Comparisons between groups for the remaining variables were performed using the two-sided Fisher exact test.

TABLE 2
Characteristics of randomized patients who
did not receive vaccination (N = 60)

| | Id-vaccine | Control | |
|---|---|---|---|
| | (N = 42) | (N = 18) | |
| Characteristic | No. (%) | No. (%) | P value^{#} |
| Age at enrollment - years | 49.6 ± 10.3* | 46.6 ± 10.8* | 0.276 |
| Male sex | 21 (50.0) | 7 (38.9) | 0.574 |
| White race group | 37 (88.1) | 14 (77.8) | 0.431 |
| ECOG Performance Status | | | 0.163 |
| 0 | 30 (71.4) | 16 (88.9) | |
| 1 | 11 (26.2) | 1 (5.5) | |
| 2 | 1 (2.4) | 1 (5.5) | |
| Histology | | | 1.000 |
| FL, grade 1 | 20 (47.6) | 8 (44.4) | |
| FL, grade 2 | 22 (52.4) | 10 (55.6) | |
| IgM isotype** | 26 (61.9) | 8 (44.4) | |
| IgG isotype** | 15 (35.7) | 8 (44.4) | |
| IgM/IgG isotype** | 0 (0.0) | 1 (5.6) | |
| IgD isotype** | 1 (2.4) | 1 (5.6) | |
| Stage | | | 0.520 |
| III | 11 (26.2) | 3 (16.7) | |
| IV | 31 (73.8) | 15 (83.3) | |
| International Prognostic Index | | | 1.000 |
| Low or low intermediate (0-2) | 36 (85.7) | 16 (88.9) | |
| High intermediate or high (3-5) | 6 (14.3) | 2 (11.1) | |
| ≦8 induction chemotherapy cycles | 22 (52.4) | 7 (38.9) | 0.405 |

*Plus-minus values are means ± SD.
**Isotypes reflect tumor biopsy isotype as determined by flow cytometry or immunohistochemistry.
^{#}Comparisons between age groups were performed with non-parametric t-tests using the normal approximation (two-sided Wilcoxon test). Comparisons between groups for the remaining variables were performed using the two-sided Fisher exact test.

TABLE 3
Summary of Grade 1 and Grade 2 Adverse Events

| Adverse Event | Id-vaccine | Control | |
|---|---|---|---|
| (Most common ≧10% in | (N = 76) | (N = 41) | |
| either group) | No. (%) | No. (%) | P value^{#} |
| Injection site reaction | 67 (88.2%) | 34 (82.9%) | 0.574 |
| Fatigue | 41 (53.9%) | 16 (39.0%) | 0.175 |
| Myalgia | 35 (46.1%) | 14 (34.1%) | 0.243 |
| Headache | 27 (35.5%) | 12 (29.3%) | 0.543 |
| Arthralgia | 25 (32.9%) | 14 (34.1%) | 1.000 |
| Infection | 16 (21.1%) | 2 (4.9%) | 0.029 |
| Nausea | 16 (21.1%) | 8 (19.5%) | 1.000 |
| Bone pain | 15 (19.7%) | 7 (17.1%) | 0.808 |
| Pruritus | 14 (18.4%) | 9 (22.0%) | 0.635 |
| Non-cardiac chest pain | 13 (17.1%) | 6 (14.6%) | 0.799 |
| Pyrexia | 13 (17.1%) | 5 (12.2%) | 0.596 |
| Dyspepsia | 12 (15.8%) | 3 (7.3%) | 0.253 |
| Flushing | 11 (14.5%) | 4 (9.8%) | 0.571 |
| Influenza like illness | 10 (13.2%) | 5 (12.2%) | 1.000 |
| Pain | 10 (13.2%) | 4 (9.8%) | 0.768 |
| Abdominal pain | 10 (13.2%) | 3 (7.3%) | 0.539 |
| Diarrhea | 10 (13.2%) | 2 (4.9%) | 0.211 |
| Sweating | 9 (11.8%) | 3 (7.3%) | 0.537 |
| Hyperglycaemia | 8 (10.5%) | 1 (2.4%) | 0.158 |

^{#}Comparisons between groups were performed with the two-sided Fisher exact test.

TABLE 4
Comparisons of baseline characteristics for defined cohorts of patients

| | | Randomized | | Randomized, | | Randomized, | | | |
|---|---|---|---|---|---|---|---|---|---|
| | | Patients | | patients | | vaccinated patients | | vaccinated patients | |
| | | randomized | | vaccinated | | with IgM isotype | | with IgG isotype | |
| | | (N = 177) | | (N = 117) | | (N = 60) | | (N = 55) | |
| | Patients enrolled | Id-vaccine | Control | Id-vaccine | Control | Id-vaccine | Control | Id-vaccine | Control |
| | (N = 234) | (N = 118) | (N = 59) | (N = 76) | (N = 41) | (N = 35) | (N = 25) | (N = 40) | (N = 15) |
| Characteristic | No. (%) | No. (%) | No. (%) | No. (%) | No. (%) | No. (%) | No. (%) | No. (%) | No. (%) |
| Age at enrollment - | 49.5 ± 10.4 | 49.8 ± 9.9 | 50.1 ± 9.8 | 49.8 ± 9.7 | 51.7 ± 9.1 | 47.4 ± 8.6 | 51.8 ± 7.3 | 52.1 ± 10.1 | 52.4 ± 11.2 |
| years (mean + SD) | | | | | | | | | |
| Male sex | 127 (54.3) | 60 (50.8) | 35 (59.3) | 39 (51.3) | 28 (68.3) | 17 (48.6) | 19 (76.0) | 21 (52.5) | 8 (53.3) |
| White race group | 208 (88.9) | 104 (88.1) | 52 (88.1) | 67 (88.2) | 38 (92.7) | 32 (91.4) | 23 (92.0) | 35 (87.5) | 14 (93.3) |
| ECOG Performance | | | | | | | | | |
| status | | | | | | | | | |
| 0 | 177 | 94 (79.7) | 46 (78.0) | 64 (84.2) | 30 (73.2) | 30 (85.7) | 20 (80.0) | 33 (82.5) | 9 (60.0) |
| 1 | 54 | 23 (19.5) | 12 (20.3) | 12 (51.8) | 11 (26.8) | 5 (14.3) | 5 (20.0) | 7 (17.5) | 6 (40.0) |
| 2 | 2 | 1 (0.8) | 1 (1.7) | — | — | — | — | — | — |
| Grade not available | 1 | — | — | — | — | — | — | — | — |
| Histology | | | | | | | | | |
| FL, grade 1 | 107 (45.7) | 54 (45.7) | 25 (42.4) | 34 (44.7) | 17 (41.5) | 20 (57.1) | 14 (56.0) | 19 (47.5) | 6 (40.0) |
| FL, grade 2 | 125 (53.4) | 64 (54.2) | 34 (57.6) | 42 (55.3) | 24 (58.5) | 15 (42.9) | 11 (45.9) | 21 (52.5) | 9 (60.0) |
| Not reported | 2 (0.9) | — | — | — | — | — | — | | |
| IgM isotype | | 61 (51.7) | 33 (56.0) | 35 (46.1) | 25 (61.0) | 35 (100.0) | 25 (100.0) | 0 (0.0) | (0.0) |
| IgG isotype | | 55 (46.6) | 23 (39.0) | 40 (52.6) | 15 (36.6) | 0 (0.0) | 0 (0.0) | 40 (100.0) | 15 (100.0) |
| IgM/IgG isotype | | 1 (0.8) | 2 (3.4) | 1 (1.3) | 1 (2.4) | — | — | — | — |
| IgD isotype | | 1 (0.8) | 1 (1.7) | 0 (0.0) | 0 (0.0) | — | — | — | — |
| Stage | | | | | | | | | |
| II | 7 (2.9) | 2 (1.7) | 1 (1.7) | 2 (2.6) | 1 (2.4) | 2 (5.7) | 1 (4.0) | 0 (0.0) | 0 (0.0) |
| III | 62 (26.5) | 40 (33.9) | 13 (22.0) | 29 (38.2) | 10 (24.4) | 9 (25.7) | 7 (28.0) | 19 (47.5) | 3 (20.0) |
| IV | 163 (69.7) | 76 (64.4) | 45 (76.3) | 45 (59.2) | 30 (73.2) | 24 (68.6) | 17 (68.0) | 21 (52.5) | 12 (80.0) |
| Not available | 2 (0.9) | — | — | — | — | — | — | — | — |
| International | | | | | | | | | |
| Prognostic Index | | | | | | | | | |
| Low or low | 205 (87.6) | 105 (89.0) | 53 (89.8) | 69 (90.8) | 37 (90.2) | 33 (94.3) | 22 (88.0) | 35 (87.5) | 14 (93.3) |
| intermediate (0-2) | | | | | | | | | |
| High intermediate or | 28 (12.0) | 13 (11.0) | 6 (10.3) | 7 (9.2) | 4 (9.8) | 2 (5.7) | 3 (12.0) | 5 (12.5) | 1 (6.67) |
| high (3-5) | | | | | | | | | |
| Not available | 1 (0.4) | — | — | — | — | — | — | — | — |
| <8 chemotherapy | 89 (38.0)* | 60 (50.8) | 29 (49.1) | 38 (50.0) | 22 (53.7) | 18 (51.4) | 12 (48.0) | 19 (47.5) | 9 (60.0) |
| cycles | | | | | | | | | |

*Data is based on the 177 randomized patients.
^{#}Comparisons between age groups were performed with non-parametric t-tests using the normal approximation (two-sided Wilcoxon test). Comparisons between groups for the remaining variables were performed using the two-sided Fisher exact test. No P values for reached statistical significance (p < 0.05).

TABLE 5
Accrual Rate by Year

| Year | Patients Enrolled No. (%) | |
|---|---|---|
| 2000 | 39 | (16.7) |
| 2001 | 35 | (14.9) |
| 2002 | 51 | (21.8) |
| 2003 | 30 | (12.8) |
| 2004 | 23 | (9.8) |
| 2005 | 22 | (9.4) |
| 2006 | 20 | (8.5) |
| 2007 | 14 | (5.9) |
| Total | 234 | |

TABLE 6
Distribution of tumor Ig isotype by treatment arm for
randomized and vaccinated patients (N = 117)

| | | Vaccine | Isotype Analysis | |
|---|---|---|---|---|
| Treatment Arm | Biopsy Isotype | Isotype | Group | N |
| Id-KLH | IgM | IgM | IgM | 29 |
| Id-KLH | IgM/IgD | IgM | IgM | 4 |
| Id-KLH | IgM/IgG | IgM | IgM | 1 |
| Id-KLH | IgM | KLH-KLH | IgM | 1 |
| Id-KLH | IgG | IgG | IgG | 35 |
| Id-KLH | IgM/IgG | IgG | IgG | 2 |
| Id-KLH | IgG | KLH-KLH | IgG | 3 |
| Id-KLH | IgM/IgG | KLH-KLH | Excluded | 1 |
| Control | IgM | KLH-KLH | IgM | 23 |
| Control | IgM/IgD | KLH-KLH | IgM | 1 |
| Control | IgM/IgA | KLH-KLH | IgM | 1 |
| Control | IgG | KLH-KLH | IgG | 13 |
| Control | IgG/IgA | KLH-KLH | IgG | 2 |
| Control | IgM/IgG | KLH-KLH | Excluded | 1 |

TABLE 7
Summary of Grade 3 and Grade 4 Adverse Events

| | Id-vaccine | | Control | |
|---|---|---|---|---|
| | (N = 76) | | (N = 41) | |
| Adverse Event | No. (%) | | No. (%) | |
| Vomiting | 1 | (1.3%) | 1 | (2.4%) |
| Urticaria | 1 | (1.3%) | 1 | (2.4%) |
| Headache | 1 | (1.3%) | 1 | (2.4%) |
| Osteonecrosis | 1 | (1.3%) | 0 | (0.0%) |
| Fatigue | 1 | (1.3%) | 0 | (0.0%) |
| injection site reaction | 1 | (1.3%) | 0 | (0.0%) |
| Myalgia | 1 | (1.3%) | 0 | (0.0%) |
| Diarrhea | 1 | (1.3%) | 0 | (0.0%) |
| Non-cardiac chest pain | 1 | (1.3%) | 0 | (0.0%) |
| Cerebral ischemia | 1 | (1.3%) | 0 | (0.0%) |
| Myocardial ischemia | 1 | (1.3%) | 0 | (0.0%) |
| Hypertension | 1 | (1.3%) | 0 | (0.0%) |
| Abdominal pain | 1 | (1.3%) | 0 | (0.0%) |
| Dyspepsia | 1 | (1.3%) | 0 | (0.0%) |
| Erythema multiforme | 1 | (1.3%) | 0 | (0.0%) |
| Acute myeloid leukemia | 1 | (1.3%) | 0 | (0.0%) |
| Induration | 1 | (1.3%) | 0 | (0.0%) |
| Dizziness | 0 | (0.0%) | 1 | (2.4%) |
| Arthralgia | 0 | (0.0%) | 1 | (2.4%) |
| Compression fracture | 0 | (0.0%) | 1 | (2.4%) |
| Dyspnea | 0 | (0.0%) | 1 | (2.4%) |
| Pain | 0 | (0.0%) | 1 | (2.4%) |
| Herpes zoster | 0 | (0.0%) | 1 | (2.4%) |
| Arrhythmia | 0 | (0.0%) | 1 | (2.4%) |
| Squamous cell carcinoma of | 0 | (0.0%) | 1 | (2.4%) |
| skin | | | | |
| Cystitis interstitial | 0 | (0.0%) | 1 | (2.4%) |
| Intervertebral disc protrusion | 0 | (0.0%) | 1 | (2.4%) |
| Total* | 17 | | 13 | |

*Between groups comparison for the overall rate of grade 3-4 adverse events was performed with the two-sided Fisher exact statistic using the total number of vaccinations administered for all patients on each arm (P = 0.331).

TABLE 8
comparison of NCI Phase 2 and Randomized phase III clinical trials with idiotype vaccine in follicular lymphoma

| | NCI Phase 2^{3,4} | NCI/Biovest | Genitope⁵ | Favrille⁶ |
|---|---|---|---|---|
| | (NCT00001512) | (NCT00091676) | (NCT00017290) | (NCT00089115) |
| Id protein in vaccine | Native protein from | Native protein from | Recombinant protein | Recombinant protein |
| formulation | heterohybridoma | heterohybridoma | from mammalian cell | from Sf9 (insect) cell |
| | | | line | line |
| Isotype of Id-vaccine | IgM or IgG | IgM or IgG | IgG | IgG |
| | (tumor-matched) | (tumor-matched) | | |
| Induction therapy | PACE | PACE | CVP | Rituximab |
| Prerequisite for | CR/CRu | CR/CRu | CR/CRu/PR | CR/CRu/PR/SD |
| vaccination | | | | |
| Randomization | Open-label | 2:1 | 2:1 | 1:1 |
| Stratification | Not applicable | IPI score 0-2 vs. 3, 4 | Not reported | Treatment-naive vs. |
| | | <8 vs. ≧8 cycles | | relapsed |
| | | | | CR/CRu/PR vs. SD |
| Primary endpoint | Induction of immune | DFS | Progression-free | Time to progression |
| | responses and | | survival | |
| | molecular-remissions | | | |
| Clinical outcome with Id- | 75% antibody | Significant | No improvement | No improvement |
| vaccine | responses and 95% T- | improvement in DFS | | |
| | cell responses; 45% | | | |
| | remain in CR after | | | |
| | median follow-up of | | | |
| | 9.2 years | | | |

NCI, National Cancer Institute;
FL, follicular lymphoma;
PACE, prednisone, doxorubicin, cyclophosphamide, etoposide;
CVP, cyclophosphamide, vincristine, prednisone;
CR, complete response,
CRu, complete response unconfirmed;
PR, partial response;
SD, stable disease,
IPI, Internatonal Prognostic Index;
ID, idiotype,;
DFS, disease-free survival.

What is claimed is:

1. (canceled)

2. A method for treating a B-cell malignancy in a subject in which the immunoglobulin isotype or isotypes exhibited by the malignancy have been predetermined, comprising administering an autologous idiotype vaccine to the subject, wherein the vaccine comprises an autologous idiotype immunoglobulin comprising at least an IgM constant region.

3. The method of claim 2, wherein the malignancy exhibits a predetermined immunoglobulin isotype or isotypes that is not an IgM isotype (a non-IgM immunoglobulin).

4. The method of claim 2, wherein the malignancy exhibits a predetermined immunoglobulin isotype or isotypes that is an IgM isotype (an IgM immunoglobulin).

5. The method of claim 3, wherein the non-IgM immunoglobulin is IgG, IgA, IgD, IgE, or any combination of two or more of the foregoing.

6. The method of claim 5, wherein the non-IgM immunoglobulin is IgG1, IgG2, IgG3, IgG4, IgA1, IgA2, IgE, IgD, or any combination of the foregoing.

7. The method of claim 2, wherein the vaccine comprises a chimeric idiotype immunoglobulin comprising at least an IgM constant region, and a variable region derived from a non-IgM immunoglobulin expressed by the malignancy.

8. The method of claim 2, wherein the vaccine comprises a chimeric idiotype immunoglobulin comprising at least an IgM constant region, and a variable region derived from an IgM immunoglobulin expressed by the malignancy.

9. The method of claim 5, wherein the chimeric idiotype immunoglobulin is produced recombinantly by introducing a genetic construct into a host cell, wherein the genetic construct comprises a nucleic acid sequence encoding the IgM constant region and a nucleic acid sequence encoding the variable region of the immunoglobulin expressed by the malignant cell wherein the isotype of the immunoglobulin is not IgM, and wherein the nucleic sequences are expressed by the host cell.

10. The method of claim 9, wherein the host cell is a mammalian cell, insect cell, bacterial cell, plant cell, viral cell, or fungal cell.

11. The method of claim 2, wherein the malignancy exhibits predetermined immunoglobulin isotypes that are mixed, and wherein the vaccine comprises an IgM idiotype immunoglobulin.

12. The method of claim 2, wherein the malignancy exhibits a predetermined immunoglobulin isotype that is only IgM, and wherein the vaccine comprises an IgM idiotype immunoglobulin.

13. The method of claim 2, wherein the vaccine comprises an idiotype immunoglobulin that is produced by hybridoma rescue fusion hybridization.

14-48. (canceled)

49. A method for selecting a treatment for a subject having a B-cell malignancy, comprising screening the patient for a heavy-chain isotype, wherein if the isotype has detectable M isotype, production of an autologous idiotype IgM vaccine is authorized and treatment of the subject with the autologous idiotype IgM vaccine can proceed; and
wherein if the subject has only a non-IgM B-cell malignancy, (a) production of a recombinant idiotype vaccine for the subject is authorized and treatment of the subject with the recombinant vaccine can proceed; or optionally (b) the subject is excluded from treatment with an idiotype vaccine and an alternative treatment with an alternative (non-idiotype vaccine) therapy is authorized and may proceed.

50. The method of claim 49, further comprising administering the autologous idiotype IgM vaccine to the subject.

51. The method of claim 49, further comprising administering the recombinant idiotype vaccine to the subject.

52. The method of claim 49, further comprising administering the alternative therapy to the subject.

53-78. (canceled)

79. An autologous idiotype vaccine comprising at least an IgM constant region.

80. (canceled)

81. The vaccine of claim 79, wherein the vaccine comprises a chimeric idiotype immunoglobulin comprising at least an IgM constant region, and a variable region derived from a non-IgM immunoglobulin expressed by a B-cell malignancy.

82. (canceled)

83. The vaccine of claim 79, wherein the vaccine comprises a chimeric idiotype immunoglobulin that is produced recombinantly by introducing a genetic construct into a host cell, wherein the genetic construct comprises a nucleic acid sequence encoding the IgM constant region and a nucleic acid sequence coding for the variable region of the immunoglobulin expressed by the malignant cell wherein the isotype of the immunoglobulin is not IgM, and wherein the nucleic sequences are expressed by the host cell.
